SEQUENCE LISTING (53 sequences)

SEQ ID NO: 1 - protein, 116 aa, synthetic construct
  REGION 1..116: note = synthetic polypeptide
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 2 - protein, 116 aa, synthetic construct
  REGION 1..116: note = synthetic polypeptide
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 116 aa, synthetic construct
  REGION 1..116: note = synthetic polypeptide
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 4 - protein, 116 aa, synthetic construct
  REGION 1..116: note = synthetic polypeptide
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 5 - protein, 116 aa, synthetic construct
  REGION 1..116: note = synthetic polypeptide
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 6 - protein, 116 aa, synthetic construct
  REGION 1..116: note = synthetic polypeptide
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 7 - protein, 111 aa, synthetic construct
  REGION 1..111: note = synthetic polypeptide
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 8 - protein, 111 aa, synthetic construct
  REGION 1..111: note = synthetic polypeptide
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELK

SEQ ID NO: 9 - protein, 111 aa, synthetic construct
  REGION 1..111: note = synthetic polypeptide
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 10 - protein, 111 aa, synthetic construct
  REGION 1..111: note = synthetic polypeptide
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 11 - protein, 111 aa, synthetic construct
  REGION 1..111: note = synthetic polypeptide
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 12 - protein, 465 aa, synthetic construct
  REGION 1..465: note = synthetic polypeptide
MGWSCIILFLVATATGVHSEVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 465 aa, synthetic construct
  REGION 1..465: note = synthetic polypeptide
MGWSCIILFLVATATGVHSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 237 aa, synthetic construct
  REGION 1..237: note = synthetic polypeptide
MGWSCIILFLVATATGVHSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 5 aa, synthetic construct
  REGION 1..5: note = synthetic polypeptide
DYGMH

SEQ ID NO: 16 - protein, 5 aa, synthetic construct
  REGION 1..5: note = synthetic polypeptide
NYGMH

SEQ ID NO: 17 - protein, 17 aa, synthetic construct
  REGION 1..17: note = synthetic polypeptide
YISSGSSTIYYADTVKG

SEQ ID NO: 18 - protein, 7 aa, synthetic construct
  REGION 1..7: note = synthetic polypeptide
RGLLLDY

SEQ ID NO: 19 - protein, 17 aa, synthetic construct
  REGION 1..17: note = synthetic polypeptide
YISSGSSTIYYADSVKG

SEQ ID NO: 20 - protein, 8 aa, synthetic construct
  REGION 1..8: note = synthetic polypeptide
GFTFSDYG

SEQ ID NO: 21 - protein, 8 aa, synthetic construct
  REGION 1..8: note = synthetic polypeptide
GFTFSNYG

SEQ ID NO: 22 - protein, 8 aa, synthetic construct
  REGION 1..8: note = synthetic polypeptide
ISSGSSTI

SEQ ID NO: 23 - protein, 9 aa, synthetic construct
  REGION 1..9: note = synthetic polypeptide
ARRGLLLDY

SEQ ID NO: 24 - protein, 15 aa, synthetic construct
  REGION 1..15: note = synthetic polypeptide
RASKSVSTSSYSYMH

SEQ ID NO: 25 - protein, 7 aa, synthetic construct
  REGION 1..7: note = synthetic polypeptide
YASYLES

SEQ ID NO: 26 - protein, 9 aa, synthetic construct
  REGION 1..9: note = synthetic polypeptide
QHSREFPWT

SEQ ID NO: 27 - protein, 15 aa, synthetic construct
  REGION 1..15: note = synthetic polypeptide
RASKSVSTSSYSYLA

SEQ ID NO: 28 - protein, 7 aa, synthetic construct
  REGION 1..7: note = synthetic polypeptide
YASYLQS

SEQ ID NO: 29 - protein, 10 aa, synthetic construct
  REGION 1..10: note = synthetic polypeptide
KSVSTSSYSY

SEQ ID NO: 30 - skipped

SEQ ID NO: 31 - protein, 7 aa, synthetic construct
  REGION 1..7: note = synthetic polypeptide
GQSSRSS

SEQ ID NO: 32 - protein, 18 aa, synthetic construct
  REGION 1..18: note = synthetic polypeptide
GQSSRSSSGGGSSGGGGS

SEQ ID NO: 33 - protein, 4 aa, synthetic construct
  REGION 1..4: note = synthetic polypeptide
GSGS

SEQ ID NO: 34 - protein, 4 aa, synthetic construct
  REGION 1..4: note = synthetic polypeptide
GGGS

SEQ ID NO: 35 - protein, 10 aa, synthetic construct
  REGION 1..10: note = synthetic polypeptide
GGGGSGGGGS

SEQ ID NO: 36 - protein, 20 aa, synthetic construct
  REGION 1..20: note = synthetic polypeptide
GGGGSGGGGSGGGGSGGGGS

SEQ ID NO: 37 - protein, 15 aa, synthetic construct
  REGION 1..15: note = synthetic polypeptide
GGGGSGGGGSGGGGS

SEQ ID NO: 38 - protein, 274 aa, synthetic construct
  REGION 1..274: note = synthetic polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 39 - protein, 541 aa, synthetic construct
  REGION 1..541: note = synthetic polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 40 - protein, 808 aa, synthetic construct
  REGION 1..808: note = synthetic polypeptide
AGIHDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSASTKGPSVFPLAPLESSGSDIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGSEVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSSLEQKLISEEDLNSAVDHHHHHH

SEQ ID NO: 41 - protein, 242 aa, synthetic construct
  REGION 1..242: note = synthetic polypeptide
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIKGGGGSGGGGSGGGGSEVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 42 - protein, 19 aa, synthetic construct
  REGION 1..19: note = synthetic polypeptide
HHHHRKKRRQRRRRHHHHH

SEQ ID NO: 43 - protein, 30 aa, synthetic construct
  REGION 1..30: note = synthetic polypeptide
MVKSKIGSWILVLFVAMWSDVGLCKKRPKP

SEQ ID NO: 44 - protein, 27 aa, synthetic construct
  REGION 1..27: note = synthetic polypeptide
GALFLGFLGAAGSTMGAWSQPKKKRKV

SEQ ID NO: 45 - DNA, 18 bp, synthetic construct
  source 1..18: mol_type = other DNA
  misc_feature 1..18: note = synthetic polynucleotide
  misc_feature 1: note = linked to lys-lys-lys
  misc_feature 1: note = pseudoisocytosine
  misc_feature 2: note = pseudoisocytosine
  misc_feature 4: note = pseudoisocytosine
  misc_feature 7: note = pseudoisocytosine
  misc_feature 9..10: note = Linked by three 8-amino-2, 6, 10-trioxaoctanoic acid, three8-amino-3,6-dioxaoctanoic acid, or three 6-aminohexanoic acidmolecules
  misc_feature 18: note = Linked to lys-lys-lys
nntnttnttttcttctcc

SEQ ID NO: 46 - DNA, 51 bp, synthetic construct
  source 1..51: mol_type = other DNA
  misc_feature 1..51: note = synthetic polynucleotide
  misc_feature 1..2: note = optional phosphorothioate internucleoside linkage
  misc_feature 2..3: note = optional phosphorothioate internucleoside linkage
  misc_feature 3..4: note = optional phosphorothioate internucleoside linkage
  misc_feature 48..49: note = optional phosphorothioate internucleoside linkage
  misc_feature 49..50: note = optional phosphorothioate internucleoside linkage
  misc_feature 50..51: note = optional phosphorothioate internucleoside linkage
gttcagcgtgtccggcgagggcgaggtgagtctatgggacccttgatgttt

SEQ ID NO: 47 - DNA, 60 bp, synthetic construct
  source 1..60: mol_type = other DNA; note = synthetic polynucleotide
  modified_base 1..2: mod_base = OTHER; note = optional phosphorothioate internucleoside linkage
  modified_base 2..3: mod_base = OTHER; note = optional phosphorothioate internucleoside linkage
  modified_base 3..4: mod_base = OTHER; note = optional phosphorothioate internucleoside linkage
  modified_base 57..58: mod_base = OTHER; note = optional phosphorothioate internucleoside linkage
  modified_base 58..59: mod_base = OTHER; note = optional phosphorothioate internucleoside linkage
  modified_base 59..60: mod_base = OTHER; note = optional phosphorothioate internucleoside linkage
aaagaataacagtgataatttctgggttaaggcaatagcaatatctctgcatataaatat

SEQ ID NO: 48 - DNA, 30 bp, synthetic construct
  source 1..30: mol_type = other DNA; note = synthetic polynucleotide
  modified_base 1: mod_base = OTHER; note = Linked to lys-lys-lys
  modified_base 1: mod_base = OTHER; note = n = pseudoisocytosine
  modified_base 5: mod_base = OTHER; note = n = pseudoisocytosine
  modified_base 9: mod_base = OTHER; note = n = pseudoisocytosine
  modified_base 11: mod_base = OTHER; note = n = pseudoisocytosine
  modified_base 12..13: mod_base = OTHER; note = Linked by three 8-amino-2, 6, 10-trioxaoctanoic acid, three8-amino-3,6-dioxaoctanoic acid, or three 6-aminohexanoic acidmolecules
  modified_base 30: mod_base = OTHER; note = Optional phosphorothiate internucleoside linkage
ntttntttntnttctctttctttcagggca

SEQ ID NO: 49 - DNA, 25 bp, synthetic construct
  source 1..25: mol_type = other DNA; note = synthetic polynucleotide
  modified_base 1: mod_base = OTHER; note = Linked to lys-lys-lys
  modified_base 1: mod_base = OTHER; note = n = pseudoisocytosine
  modified_base 2: mod_base = OTHER; note = n = pseudoisocytosine
  modified_base 4: mod_base = OTHER; note = n = pseudoisocytosine
  modified_base 7: mod_base = OTHER; note = n = pseudoisocytosine
  modified_base 7..8: mod_base = OTHER; note = Linked by three 8-amino-2, 6, 10-trioxaoctanoic acid, three8-amino-3,6-dioxaoctanoic acid, or three 6-aminohexanoic acidmolecules
  modified_base 25: mod_base = OTHER; note = Linked to lys-lys-lys
nntnttncttctccacaggagtcag

SEQ ID NO: 50 - DNA, 65 bp, synthetic construct
  source 1..65: mol_type = other DNA; note = synthetic polynucleotide
  modified_base 1..2: mod_base = OTHER; note = optional phosphorothioate internucleoside linkage
  modified_base 2..3: mod_base = OTHER; note = optional phosphorothioate internucleoside linkage
  modified_base 3..4: mod_base = OTHER; note = optional phosphorothioate internucleoside linkage
  modified_base 62..63: mod_base = OTHER; note = optional phosphorothioate internucleoside linkage
  modified_base 63..64: mod_base = OTHER; note = optional phosphorothioate internucleoside linkage
  modified_base 64..65: mod_base = OTHER; note = optional phosphorothioate internucleoside linkage
ttgccccacagggcagtaacggcagacttctcctcaggagtcaggtgcaccatggtgtctgtttg

SEQ ID NO: 51 - DNA, 25 bp, synthetic construct
  source 1..25: mol_type = other DNA; note = synthetic polynucleotide
  modified_base 1: mod_base = OTHER; note = Linked to lys-lys-lys
  modified_base 3: mod_base = OTHER; note = n = pseudoisocytosine
  modified_base 4: mod_base = OTHER; note = n = pseudoisocytosine
  modified_base 6: mod_base = OTHER; note = n = pseudoisocytosine
  modified_base 7..8: mod_base = OTHER; note = Linked by three 8-amino-2, 6, 10-trioxaoctanoic acid, three8-amino-3,6-dioxaoctanoic acid, or three 6-aminohexanoic acidmolecules
  modified_base 25: mod_base = OTHER; note = Linked to lys-lys-lys
ttnntnttctccttaaacctgtctt

SEQ ID NO: 52 - DNA, 127 bp, synthetic construct
  source 1..127: mol_type = other DNA; note = synthetic polynucleotide
  modified_base 1..2: mod_base = OTHER; note = optional phosphorothioate internucleoside linkage
  modified_base 2..3: mod_base = OTHER; note = optional phosphorothioate internucleoside linkage
  modified_base 3..4: mod_base = OTHER; note = optional phosphorothioate internucleoside linkage
  modified_base 124..125: mod_base = OTHER; note = optional phosphorothioate internucleoside linkage
  modified_base 125..126: mod_base = OTHER; note = optional phosphorothioate internucleoside linkage
  modified_base 126..127: mod_base = OTHER; note = optional phosphorothioate internucleoside linkage
gccacctacggcaagctgaccctgaagttcatctgcaccaccggcaagctgccggtgccctggcccaccctcgtgaccaccctgacctacggcgtgcagtgcttcagccgctaccccgaccacatga

SEQ ID NO: 53 - RNA, 20 nt, synthetic construct
  misc_feature 1..20: note = synthetic polynucleotide
  source 1..20: mol_type = other RNA
gctgaagcactgcacgccat

(19) United States
(12) Patent Application Publication
(10) Pub. No.: US 2026/0048118 A1
(43) Pub. Date: Feb. 19, 2026

(54) COMPOSITIONS AND METHODS FOR ENHANCING TRIPLEX AND NUCLEASE-BASED GENE EDITING

(71) Applicant: Yale University, New Haven, CT (US)

(72) Inventors: Elias Quijano, Durham, CT (US); Adele Ricciardi, New Haven, CT (US); Raman Bahal, Glastonbury, CT (US); Audrey Turchick, Chelmsford, MA (US); Nicholas Economos, New Haven, CT (US); W. Mark Saltzman, New Haven, CT (US); Peter Glazer, Guilford, CT (US)

(73) Assignee: Yale University, New Haven, CT (US)

(21) Appl. No.: 19/277,303

(22) Filed: Jul. 22, 2025

Related U.S. Application Data
(63) Continuation of application No. 17/272,151, now abandoned.
(60) Provisional application No. 62/725,852, filed on Aug. 31, 2018.

(51) Int. Cl. A61K 39/395; A61K 9/51; A61K 31/7088; A61K 35/28; A61K 38/14; A61K 38/46; A61K 39/00; A61K 48/00; A61P 7/00

(52) U.S. Cl. CPC A61K 39/39533; A61K 9/5153; A61K 31/7088; A61K 35/28; A61K 38/14; A61K 38/465; A61P 7/00; A61K 2039/545; A61K 48/00

(57) ABSTRACT

Compositions for improved gene editing and methods of use thereof are disclosed. In a preferred method, gene editing involves use of a cell-penetrating anti-DNA antibody, such as 3E10, as a potentiating agent to enhance gene editing by nucleases and triplex forming oligonucleotides. Genomic modification occurs at a higher frequency when cells are contacted with the potentiating agent and nuclease or triplex forming oligonucleotide, as compared to the absence of the potentiating agent. The methods are suitable for both ex vivo and in vivo approaches to gene editing and are useful for treating a subject with a genetic disease or disorder. Nanoparticle compositions for intracellular delivery of the gene editing compositions are provided and are particularly advantageous for use with in vivo applications.

### CROSS-REFERENCE TO RELATED APPLICATION

This application is a continuation of U.S. application Ser. No. 17/272,151, which is the U.S. National Phase of International Application No. PCT/US2019/048962, filed on Aug. 30, 2019, which claims the priority benefit of U.S. Provisional Application No. 62/725,852, filed Aug. 31, 2018, each of which is specifically incorporated by reference herein in its entirety.

### STATEMENT REGARDING FEDERALLY SPONSORED RESEARCH

This invention was made with government support under CA197574 and CA168733 awarded by the National Institutes of Health. The government has certain rights in the invention.

### REFERENCE TO THE SEQUENCE LISTING

The content of the electronically submitted sequence listing (Name: 2681_1230003_Sequencelisting_ST26; Size: 96,521 bytes; and Date of Creation: Nov. 3, 2025) is hereby incorporated by reference in its entirety.

### FIELD OF THE INVENTION

The invention is generally related to the field of gene editing technology, and more particularly to methods of using cell-penetrating antibodies to improve triplex-forming oligonucleotide- and nuclease-mediated gene editing.

### BACKGROUND OF THE INVENTION

Gene editing provides an attractive strategy for treatment of inherited genetic disorders such as, for example, sickle cell anemia and β-thalassemia. Genes can be selectively edited by several methods, including targeted nucleases such as zinc finger nucleases (ZFNs) (Haendel, et al., Gene Ther., 11:28-37 (2011)) and CRISPRs (Yin, et al., Nat. Biotechnol., 32:551-553 (2014)), short fragment homologous recombination (SFHR) (Goncz, et al., Oligonucleotides, 16:213-224 (2006)), or triplex-forming oligonucleotides (TFOs) (Vasquez, et al., Science, 290:530-533 (2000)). It is generally thought that a DNA break in a target gene is needed for high efficiency gene editing with a donor DNA. Hence, there has been widespread focus on targeted nucleases such as CRISPR/Cas9 technology because of its ease of use and facile reagent design (Doudna, et al., Science, 346:1258096 (2014)). However, like ZFNs, the CRISPR approach introduces an active nuclease into cells, which can lead to off-target cleavage in the genome (Cradick, et al., Nucleic Acids Res., 41:9584-9592 (2013)), a problem that so far has not been eliminated.

Alternatives have been developed such as triplex-forming peptide nucleic acid (PNA) oligomers which recruit the cell's endogenous DNA repair systems to initiate site-specific modification of the genome when single-stranded “donor DNAs” are co-delivered as templates (Rogers, et al., Proc. Natl. Acad. Sci. USA, 99:16695-16700 (2002)).

Historically however, the efficiency of gene modification could be low, especially in the context of CRISPR/Cas-mediated editing in primary stem cells. For example, in an attempt to correct the CFTR locus in cystic fibrosis patient derived stem cells, approximately 0.3% of treated organoids (3 to 6/1400) had the desired modification (Schwank, et al., Cell Stem Cell., 13:653-658 (2013)).

Accordingly, there remains a need for compositions and methods for improved gene editing.

It is therefore an object of the invention to provide gene editing potentiating agents and methods for achieving an increased frequency of gene modification.

It is another object of the invention to provide methods for achieving on-target modification with reduced or low off-target modification.

It is a further object of the invention to provide compositions and methods for gene modification that improve one or more symptoms of a disease or disorder in a subject.

### SUMMARY OF THE INVENTION

Compositions for enhancing targeted gene editing and methods of use thereof are disclosed. Disclosed are methods of gene editing utilizing a gene editing composition such as triplex-forming oligonucleotides, CRISPR, zinc finger nucleases, TALENS, or others, in combination with a gene editing potentiating agent such as a cell-penetrating anti-DNA antibody.

An exemplary method of modifying the genome of a cell can include contacting the cell with an effective amount of (i) a gene editing potentiating agent, and (ii) a gene editing technology that can induce genomic modification of the cell (e.g., triplex-forming molecules, pseudocomplementary oligonucleotides, a CRISPR system, zinc finger nucleases (ZFN), and transcription activator-like effector nucleases (TALEN)). In the foregoing method, genomic modification occurs at a higher frequency in a population of cells contacted with both (i) and (ii), than in an equivalent population contacted with (ii) in the absence of (i). Preferred gene editing technologies include a triplex forming molecule, such as a peptide nucleic acid (PNA), and a CRISPR system such as CRISPR/Cas9 D10A nickase.

A preferred gene editing potentiating agent is a cell-penetrating anti-DNA antibody which is transported into the cytoplasm and/or nucleus of the cell without the aid of a carrier or conjugate. In some embodiments, the cell-penetrating anti-DNA antibody is isolated or derived from a subject with systemic lupus erythematous or an animal model thereof (such as a mouse or rabbit). In a preferred embodiment, the cell-penetrating anti-DNA antibody is the monoclonal anti-DNA antibody 3E10, or a variant, fragment (e.g., cell-penetrating fragment), or humanized form thereof that binds the same epitope(s) as 3E10. A particularly preferred variant is a 3E10 variant incorporating a D31N substitution in the heavy chain. The cell-penetrating anti-DNA antibody may have the same or different epitope specificity as monoclonal antibody 3E10 produced by ATCC No. PTA 2439 hybridoma.

In some embodiments, the antibody has
(i) the CDRs of any one of SEQ ID NO: 1-6, 12, or 13 in combination with the CDRs of any one of SEQ ID NO:7-11, or 15;
(ii) first, second, and third heavy chain CDRs selected from SEQ ID NOS: 15-23 in combination with first, second and third light chain CDRs selected from SEQ ID NOS: 24-30;
(iii) humanized forms of (i) or (ii);
(iv) a heavy chain comprising an amino acid sequence comprising at least 85% sequence identity to any one of SEQ ID NO:1 or 2 in combination with a light chain comprising an amino acid sequence comprising at least 85% sequence identity to SEQ ID NO:7 or 8;
(v) a humanized form or (iv); or
(vi) a heavy chain comprising an amino acid sequence comprising at least 85% sequence identity to any one of SEQ ID NO:3-6 in combination with a light chain comprising an amino acid sequence comprising at least 85% sequence identity to SEQ ID NO:9-11.

Preferably, the antibody can bind directly to RAD51. In some embodiments, the anti-DNA antibody has the paratope of monoclonal antibody 3E10. The anti-DNA antibody may be a single chain variable fragment of an anti-DNA antibody, or conservative variant thereof. For example, the anti-DNA antibody can be a monovalent, divalent, or multivalent single chain variable fragment of 3E10 (3E10 Fv), or a variant, for example a conservative variant, thereof. In some embodiments, the anti-DNA antibody is a monovalent, divalent, or multivalent single chain variable fragment of 3E10 (3E10 Fv) incorporating a D31N substitution in the heavy chain.

The method can further include contacting the cells with a donor oligonucleotide including, for example, a sequence that corrects or induces a mutation(s) in the cell's genome by insertion or recombination of the donor induced or enhanced by the gene editing technology. The donor oligonucleotide (e.g., DNA) may be single stranded or double stranded. Preferably, the donor oligonucleotide is single stranded DNA. The potentiating agent, gene editing technology, and/or donor oligonucleotide can be contacted with the cell in any order.

In some embodiments, the cell's genome has a mutation underlying a disease or disorder, for example a genetic disorder such as hemophilia, muscular dystrophy, globinopathies, cystic fibrosis, xeroderma pigmentosum, lysosomal storage diseases, immune deficiency syndromes such as X-linked severe combined immunodeficiency and ADA deficiency, tyrosinemia, Fanconi anemia, the red cell disorder spherocytosis, alpha-1-anti-trypsin deficiency, Wilson's disease, Leber's hereditary optic neuropathy, or chronic granulomatous disorder. The globinopathy can be sickle cell anemia or beta-thalassemia. The lysosomal storage disease can be Gaucher's disease, Fabry disease, or Hurler syndrome. In some embodiments, the method induces a mutation that reduces HIV infection, for example, by reducing an activity of a cell surface receptor that facilitates entry of HIV into the cell.

In some embodiments, the cells (e.g., hematopoietic stem cells) are contacted ex vivo and the cells may further be administered to a subject in need thereof. The cells may be administered to the subject in an effective amount to treat one or more symptoms of a disease or disorder.

In other embodiments, the cells are contacted in vivo following administration of the potentiating agent, gene editing technology, and optionally the donor oligonucleotide to a subject. Each of the foregoing can be in the same or different pharmaceutical compositions and can be administered to the subject in any order. In preferred embodiments, the compositions induce or enhance in vivo gene modification in an effective amount to reduce one or more symptoms of the disease or disorder in the subject.

Any of the disclosed compositions including potentiating agent, gene editing technology, and/or donor oligonucleotide can be packaged together or separately in nanoparticles. The nanoparticles may be formed from polyhydroxy acids. In preferred embodiments, the nanoparticles include poly(lactic-co-glycolic acid) (PLGA) alone or in a blend with poly(beta-amino) esters (PBAEs). The nanoparticles may be prepared by double emulsion or nanoprecipitation. In some embodiments, the gene editing technology, the donor oligonucleotide or a combination thereof are complexed with a polycation prior to preparation of the nanoparticles.

Functional molecules such as targeting moieties, cell penetrating peptides, or a combination thereof can be associated with, linked, conjugated, or otherwise attached directly or indirectly to the potentiating agent, the gene editing technology, the nanoparticle, or a combination thereof.

### BRIEF DESCRIPTION OF THE DRAWINGS

FIG. 1A is a bar graph showing PNA/DNA mediated gene correction of the IVS2-654 (C->T) mutation within the β-globin/GFP fusion gene in MEFs treated with Rad51 siRNA or 3E10. FIGS. 1B and 1C are box plots showing the frequency of in vivo gene editing in bone marrow-(1B) and spleen-derived (1C) CD117+ cells from β-globin/GFP transgenic mice treated with 3E10.

FIG. 2 is a bar graph showing the percentage of gene editing following treatment of MEFs from Townes mice with PNA/DNA-containing nanoparticles with or without the 3E10 antibody.

FIG. 3A is a schematic representation of binding site positions of tcPNAs 1, 2, and 3 targeting the beta globin gene in the vicinity of the SCD mutation. FIG. 3B is a bar graph showing the percentage of gene editing in bone marrow cells from Townes mice treated with tcPNA2A/donor DNA-containing nanoparticles with or without the 3E10 antibody.

FIG. 4 is a box plot showing the percentage of gene editing in bone marrow cells following in vivo treatment of Townes mice with PNA/donor DNA-containing nanoparticles with or without the 3E10 antibody.

FIG. 5 is a bar graph showing the percentage of gene editing in SC-1 cells treated with PNA/DNA-containing nanoparticles with or without the 3E10 antibody.

FIGS. 6A and 6B are bar graphs showing the percentage of Cas9-mediated gene editing in K562 BFP/GFP reporter cells treated with or without the 3E10 antibody in the presence of CRISPR/Cas9 WT (6A) and CRISPR/Cas9 D10A nickase (6B).

### DETAILED DESCRIPTION OF THE INVENTION

### I. Definitions

As used herein, the term “single chain Fv” or “scFv” as used herein means a single chain variable fragment that includes a light chain variable region (VL) and a heavy chain variable region (VH) in a single polypeptide chain joined by a linker which enables the scFv to form the desired structure for antigen binding (i.e., for the VH and VL of the single polypeptide chain to associate with one another to form a Fv). The VL and VH regions may be derived from the parent antibody or may be chemically or recombinantly synthesized.

As used herein, the term “variable region” is intended to distinguish such domain of the immunoglobulin from domains that are broadly shared by antibodies (such as an antibody Fc domain). The variable region includes a “hypervariable region” whose residues are responsible for antigen binding. The hypervariable region includes amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917).

As used herein, the term “Framework Region” or “FR” residues are those variable domain residues other than the hypervariable region residues as herein defined.

As used herein, the term “antibody” refers to natural or synthetic antibodies that bind a target antigen. The term includes polyclonal and monoclonal antibodies. In addition to intact immunoglobulin molecules, also included in the term “antibodies” are binding proteins, fragments, and polymers of those immunoglobulin molecules, and human or humanized versions of immunoglobulin molecules that bind the target antigen.

As used herein, the term “cell-penetrating antibody” refers to an immunoglobulin protein, fragment, variant thereof, or fusion protein based thereon that is transported into the cytoplasm and/or nucleus of living mammalian cells. The “cell-penetrating anti-DNA antibody” specifically binds DNA (e.g., single-stranded and/or double-stranded DNA). In some embodiments, the antibody is transported into the cytoplasm of the cells without the aid of a carrier or conjugate. In other embodiments, the antibody is conjugated to a cell-penetrating moiety, such as a cell penetrating peptide. In some embodiments, the cell-penetrating antibody is transported in the nucleus with or without a carrier or conjugate.

In addition to intact immunoglobulin molecules, also included in the term “antibodies” are fragments, binding proteins, and polymers of immunoglobulin molecules, chimeric antibodies containing sequences from more than one species, class, or subclass of immunoglobulin, such as human or humanized antibodies, and recombinant proteins containing a least the idiotype of an immunoglobulin that specifically binds DNA. The antibodies can be tested for their desired activity using the in vitro assays described herein, or by analogous methods, after which their in vivo therapeutic activities are tested according to known clinical testing methods.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala:Gly, Ser), (Arg:Lys), (Asn:Gln, His), (Asp:Glu, Cys, Ser), (Gln:Asn), (Glu:Asp), (Gly:Ala), (His:Asn, Gln), (Ile:Leu, Val), (Leu:Ile, Val), (Lys:Arg), (Met:Leu, Tyr), (Ser:Thr), (Thr:Ser), (Tip:Tyr), (Tyr:Trp, Phe), and (Val:Ile, Leu). Embodiments of this disclosure thus contemplate functional or biological equivalents of a polypeptide as set forth above. In particular, embodiments of the polypeptides can include variants having about 50%, 60%, 70%, 80%, 90%, 95%, 96%, 97%, 98%, 99%, or more sequence identity to the polypeptide of interest.

As used herein, the term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or includes a certain % sequence identity to, with, or against a given sequence D) is calculated as follows:

100 times the fraction W/Z,
where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, the term “specifically binds” refers to the binding of an antibody to its cognate antigen (for example, DNA) while not significantly binding to other antigens. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

As used herein, the term “monoclonal antibody” or “MAb” refers to an antibody obtained from a substantially homogeneous population of antibodies, i.e., the individual antibodies within the population are identical except for possible naturally occurring mutations that may be present in a small subset of the antibody molecules.

As used herein a “gene editing potentiating factor” or “gene editing potentiating agent” or “potentiating factor or “potentiating agent” refers to a compound that increases the efficacy of editing (e.g., mutation, including insertion, deletion, substitution, etc.) of a gene, genome, or other nucleic acid by a gene editing technology relative to use of the gene editing technology in the absence of the compound.

As used herein, the term “subject” means any individual who is the target of administration. The subject can be a vertebrate, for example, a mammal. Thus, the subject can be a human. The term does not denote a particular age or sex.

As used herein, the terms “effective amount” or “therapeutically effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “pharmaceutically acceptable” refers to a material that is not biologically or otherwise undesirable, i.e., the material may be administered to a subject without causing any undesirable biological effects or interacting in a deleterious manner with any of the other components of the pharmaceutical composition in which it is contained.

As used herein, the term “carrier” or “excipient” refers to an organic or inorganic ingredient, natural or synthetic inactive ingredient in a formulation, with which one or more active ingredients are combined. The carrier or excipient would naturally be selected to minimize any degradation of the active ingredient and to minimize any adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, “targeting moiety” is a substance which can direct a nanoparticle to a receptor site on a selected cell or tissue type, can serve as an attachment molecule, or serve to couple or attach another molecule. As used herein, “direct” refers to causing a molecule to preferentially attach to a selected cell or tissue type. This can be used to direct cellular materials, molecules, or drugs, as discussed below.

As used herein, the term “inhibit” or “reduce” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, a “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from a nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid sequence, and then expressing the nucleic acid in an appropriate host cell under conditions in which the fusion protein is produced.

As used herein, the term “small molecule” as used herein, generally refers to an organic molecule that is less than about 2000 g/mol in molecular weight, less than about 1500 g/mol, less than about 1000 g/mol, less than about 800 g/mol, or less than about 500 g/mol. Small molecules are non-polymeric and/or non-oligomeric.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Gene Editing Potentiating Agents

Several methods have been developed to mediate gene editing. These methods include the use of Zinc Finger Nucleases, Talens, Meganucleases, CRISPR/Cas9, and triplex-forming Peptide Nucleic Acids (PNAs) (Maeder, et al., Mol. Ther., 24 (3): 430-46 (2016); Quijano, et al., Yale J. Biol. Med., 90 (4): 583-598 (2017)). These approaches either make a direct cut at the target site DNA (nucleases), or they bind to the target gene and trigger the cells endogenous repair pathways (e.g., PNAs), which secondarily leads to strand breaks. Common among these methods, the gene editing information is carried by single-stranded or double-stranded oligonucleotides, or donor DNAs, that are co-administered to the cell or animal with the nuclease or the PNA. It is generally thought that a DNA strand break in the target site is needed to enable high efficiency gene editing with a donor DNA.

In early work with DNA triplex-forming oligonucleotides (TFOs), it was observed that RAD51, a factor implicated in homology search and strand invasion in homology-directed repair processes, was required for TFO-induced gene editing (Bahal, et al., Nat. Commun., 7:13304 (2016)). It has now been discovered that RAD51 is, in contrast, not required for PNA-mediated gene editing (through experiments using co-delivered PNAs/donor DNAs in combination with anti-RAD51 siRNAs). Moreover, it has been discovered that knockdown of RAD51 actually boosts the efficiency of editing, as measured by allele-specific PCR.

The experiments described in the Examples also show that 3E10, a cell-penetrating anti-DNA antibody that binds to and inhibits RAD51, stimulates gene editing by PNAs/donor DNAs in mouse and human cells in culture, and in mice in vivo. 3E10 is also shown to enhance gene editing by the D10A nickase version of CRISPR/Cas9 in combination with a donor DNA.

Accordingly, compositions and methods of increasing the efficacy of a gene editing technology, such as, a triplex-forming PNA and donor DNA (optionally in a nanoparticle composition), or a CRISPR/Cas9 system (e.g., CRISPR/Cas9 D10A nickase) and donor DNA are provided. The disclosed methods typically include contacting cells with both a potentiating agent and a gene editing technology. Exemplary potentiating agents and gene editing technologies are provided. The potentiating agent and gene editing technology can be part of the same or different compositions.

In some embodiments, potentiating agents can engage one or more endogenous high fidelity DNA repair pathways, or inhibit/modulate error prone (i.e. low fidelity) DNA repair pathways. Potentiating agents include, for example, modulators of DNA damage and/or DNA repair factors, modulators of homologous recombination factors, cell adhesion modulators, cell cycle modulators, cell proliferation modulators, and stem cell mobilizers. The potentiating factor may modulate (e.g., alter, inhibit, promote, compete with) one or more endogenous high fidelity DNA repair pathways or inhibit/modulate error prone (i.e. low fidelity) DNA repair pathways. In preferred embodiments, the potentiating factor may be an inhibitor of a DNA damage, DNA repair, or homologous recombination factor. In more preferred embodiments, the potentiating factor may be an inhibitor of RAD51.

For example, an inhibitor of a DNA damage and/or DNA repair factor may be used as a potentiating agent. An inhibitor of a homologous recombination factor may be used as a potentiating agent.

Cells repair DNA breaks mainly through endogenous non-homologous end joining (NHEJ) DNA-repair, the predominant but error-prone pathway that can introduce or delete nucleotides at the DNA-break region. NHEJ is therefore amenable to permanent silencing of target genes. Alternatively, cells can also repair double-strand breaks by homology-directed repair (HDR), a more accurate mechanism involving homologous recombination in the presence of a template DNA strand. Typically, targeted genome editing is directed to correction of a mutated sequence in a genome by replacing the mutated sequence with a corrective sequence provided by a template/donor DNA. As such, there is ongoing effort in the field to identify and utilize mechanisms that favor homologous recombination of a template/donor DNA to enhance efficiency of targeted genome editing. Modulating the expression and/or activity of factors involved in DNA repair is a promising approach to enhance precision genome engineering.

The term “DNA repair” refers to a collection of processes by which a cell identifies and corrects damage to DNA molecules. Single-strand defects are repaired by base excision repair (BER), nucleotide excision repair (NER), or mismatch repair (MMR). Double-strand breaks are repaired by non-homologous end joining (NHEJ), microhomology-mediated end joining (MMEJ), or homologous recombination. After DNA damage, cell cycle checkpoints are activated, which pause the cell cycle to give the cell time to repair the damage before continuing to divide. Checkpoint mediator proteins include BRCA1, MDC1, 53BP1, p53, ATM, ATR, CHK1, CHK2, and p21. Accordingly, a factor involved in any of the above-mentioned processes, including BER, NER, MMR, NHEJ, MMEJ, homologous recombination, or DNA synthesis and the like, may be described as a DNA damage and/or DNA repair factor.

Non-limiting examples of DNA damage, DNA repair, DNA synthesis, or homologous recombination factors include XRCC1, ADPRT (PARP-1), ADPRTL2, (PARP-2), POLYMERASE BETA, CTPS, MLH1, MSH2, FANCD2, PMS2, p53, p21, PTEN, RPA, RPAI, RPA2, RPA3, XPD, ERCC1, XPF, MMS19, RAD51, RAD51B, RAD51C, RAD51D, DMC1, XRCCR, XRCC3, BRCA1, BRCA2, PALB2, RAD52, RAD54, RAD50, MREU, NB51, WRN, BLM, KU70, KU80, ATM, ATR CPIK1, CHK2, FANCA, FANCB, FANCC, FANCD1, FANCD2, FANCE, FANCF, FANCG, FANCC, FANCD1, FANCD2, FANCE, FANCF, FANCG, RAD1, and RAD9. In a preferred embodiment, the DNA damage factor or DNA repair factor is RAD51.

RAD51 recombinase, an ortholog of E. coli RecA, is a key protein in homologous recombination in mammalian cells. RAD51 promotes the repair of double-strand breaks, the most harmful type of DNA lesion. Double-strand breaks can be induced by various chemical agents and ionizing radiation, and are also formed during the repair of inter-strand crosslinks. Once double-strand breaks are formed, they are processed first by exonucleases to generate extensive 3′ single-stranded DNA (ssDNA) tails (Cejka et al., Nature., 467 (7311): 112-16 (2010); Mimitou & Symington, DNA Repair., 8 (9): 983-95 (2009)). These tracks of ssDNA rapidly become coated by single strand DNA-binding protein, RPA, which is ultimately displaced from the ssDNA by RAD51. RAD51 has ATP-dependent DNA binding activity, and so binds the ssDNA tails, and multimerizes to form helical nucleoprotein filaments that promote search for homologous dsDNA sequences (Kowalczykowski, Nature., 453 (7194): 463-6 (2008)). The ability of RAD51 to displace RPA on ssDNA in cells requires several mediator proteins, which include BRCA2, RAD52, the RAD51 paralog complexes, and other proteins (Thompson & Schild, Mutat Res., 477:131-53 (2001)). Once homologous dsDNA sequences are found, RAD51 promotes DNA strand exchange between the ssDNA that resides within the filament and homologous dsDNA, i.e., an invasion of ssDNA into homologous DNA duplex that results in the displacement of the identical ssDNA from the duplex and formation of a joint molecule. Joint molecules, key intermediates of DSB repair, provide both the template and the primer for DNA repair synthesis that is required for double-strand break repair (Paques & Haber, Microbiol. Mol. Biol. Rev., 63 (2): 349-404 (1999)).

By promoting DNA strand exchange, RAD51 plays a key role in homologous recombination. The protein is evolutionarily conserved from bacteriophages to mammals. In all organisms, RAD51 orthologs play an important role in DNA repair and homologous recombination (Krough & Symington, Annu. Rev. Genet., 38:233-71 (2004); Helleday et al., DNA Repair., 6 (7): 923-35 (2007); Huang et al., Proc. Natl. Acad. Sci. USA., 93 (10): 4827-32 (1996)).

In preferred embodiments, the potentiating agent is one that antagonizes or reduces expression and/or activity of RAD51, XRCC4, or a combination thereof. For example, in some embodiments, the potentiating agent is a RAD51 and/or XRCC4 inhibitor. Non-limiting examples of potentiating agents include, ribozymes, triplex-forming molecules, siRNAs, shRNAs, miRNAs, aptamers, antisense oligonucleotides, small molecules, and antibodies.

Methods for designing and producing any of the foregoing factors are well-known in the art and can be used. For example, predesigned anti-RAD51 siRNAs are commercially available through Dharmacon (as described in the Examples) and may be used as potentiating agents. Likewise, anti-XRCC4 siRNAs, shRNAs and miRNAs are known in the art and are readily available. Further, small molecule inhibitors of XRCC4 and RAD51 are known in the art (e.g., Jekimovs, et al., Front. Oncol., 4:86 (2014)) and can be used as potentiating agents in accordance with the disclosed methods.

In some embodiments, the potentiating agent is a cell-penetrating antibody. Although the cell-penetrating molecules are generally referred to herein as “cell-penetrating antibodies,” it will be appreciated that fragments and binding proteins, including antigen-binding fragments, variants, and fusion proteins such as scFv, di-scFv, tri-scFv, and other single chain variable fragments, and other cell-penetrating molecules disclosed herein are encompassed by the phrase and also expressly provided for use in compositions and methods disclosed herein.

Cell-penetrating antibodies for use in the compositions and methods may be anti-DNA antibodies. The cell-penetrating antibody may bind single stranded DNA and/or double stranded DNA. The cell-penetrating antibody may be an anti-RNA antibody (e.g., the antibody specifically binds RNA).

Autoantibodies to double-stranded deoxyribonucleic acid (dsDNA) are frequently identified in the serum of patients with systemic lupus erythematosus (SLE) and are often implicated in disease pathogenesis. Therefore, in some embodiments, cell-penetrating antibodies (e.g., cell-penetrating anti-DNA antibodies) can be derived or isolated from patients with SLE or animal models of SLE.

In preferred embodiments, the anti-DNA antibodies are monoclonal antibodies, or antigen binding fragments or variants thereof. In some embodiments, the anti-DNA antibodies are conjugated to a cell-penetrating moiety, such as a cell penetrating peptide to facilitate entry into the cell and transport to the cytoplasm and/or nucleus. Examples of cell penetrating peptides include, but are not limited to, Polyarginine (e.g., R₉), Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol). In other embodiments, the antibody is modified using TransMabs™ technology (InNexus Biotech., Inc., Vancouver, BC).

In preferred embodiments, the anti-DNA antibody is transported into the cytoplasm and/or nucleus of the cells without the aid of a carrier or conjugate. For example, the monoclonal antibody 3E10 and active fragments thereof that are transported in vivo to the nucleus of mammalian cells without cytotoxic effect are disclosed in U.S. Pat. Nos. 4,812,397 and 7,189,396 to Richard Weisbart. Briefly, the antibodies may be prepared by fusing spleen cells from a host having elevated serum levels of anti-DNA antibodies (e.g., MRL/1pr mice) with myeloma cells in accordance with known techniques or by transforming the spleen cells with an appropriate transforming vector to immortalize the cells. The cells may be cultured in a selective medium and screened to select antibodies that bind DNA.

In some embodiments, the cell-penetrating antibody may bind and/or inhibit Rad51. See for example, the cell-penetrating antibody described in Turchick, et al., Nucleic Acids Res., 45 (20): 11782-11799 (2017).

Antibodies that can be used in the compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies. Therefore, the antibodies typically contain at least the CDRs necessary to maintain DNA binding and/or interfere with DNA repair.

### A. 3E10 Sequences

In some embodiments, the cell-penetrating anti-DNA antibody is the monoclonal anti-DNA antibody 3E10, or a variant, derivative, fragment, or humanized form thereof that binds the same or different epitope(s) as 3E10. Thus, the cell-penetrating anti-DNA antibody may have the same or different epitope specificity as monoclonal antibody 3E10 produced by ATCC No. PTA 2439 hybridoma. The anti-DNA antibody can have the paratope of monoclonal antibody 3E10. The anti-DNA antibody can be a single chain variable fragment of an anti-DNA antibody, or conservative variant thereof. For example, the anti-DNA antibody can be a single chain variable fragment of 3E10 (3E10 Fv), or a variant thereof.

Amino acid sequences of monoclonal antibody 3E10 are known in the art. For example, sequences of the 3E10 heavy and light chains are provided below, where single underlining indicates the CDR regions identified according to the Kabat system, and in SEQ ID NOS: 12-14 italics indicates the variable regions and double underlining indicates the signal peptide. CDRs according to the IMGT system are also provided.

### 1. 3E10 Heavy Chain

In some embodiments, a heavy chain variable region of 3E10 is:

EVQLVESGGGLVKPGGSRKLSCAASGFTFSDYGMHWVRQAPEKGLEWVA
YISSGSSTIYYADTVKGRFTISRDNAKNTLFLQMTSLRSEDTAMYYCAR
RGLLLDYWGQGTTLTVSS
(SEQ ID NO: 1; Zack, et al.,
Immunology and Cell Biology, 72:513-520 (1994);
GenBank: L16981.1-Mouse Ig rearranged L-chain
gene, partial cds; and GenBank: AAA65679.1-
immunoglobulin heavy chain, partial [Mus
musculus]).

In some embodiments, a 3E10 heavy chain is expressed as

(3E10 WT Heavy Chain; SEQ ID NO: 12)
MGWSCIILFLVATATGVHSEVQLVESGGGLVKPGGSRKLSCAASGFTFS
DYGMHWVRQAPERGLEWVAYISSGSSTIYYADTVKGRFTISRDNAKNTL
FLQMTSLRSEDTAMYYCARRGLLLDYWGQGTTLTVSAASTKGPSVFPLA
PSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK.

Variants of the 3E10 antibody which incorporate mutations into the wild type sequence are also known in the art, as disclosed for example, in Zack, et al., J. Immunol., 157 (5): 2082-8 (1996). For example, amino acid position 31 of the heavy chain variable region of 3E10 has been determined to be influential in the ability of the antibody and fragments thereof to penetrate nuclei and bind to DNA (bolded in SEQ ID NOS: 1, 2 and 13). A D31N mutation (bolded in SEQ ID NOS: 2 and 13) in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)).

In some embodiments, an amino acid sequence for a preferred variant of a heavy chain variable region of 3E10 is:

(SEQ ID NO: 2)
EVQLVESGGGLVKPGGSRKLSCAASGFTFSNYGMHWVRQAPEKGLEWVA
YISSGSSTIYYADTVKGRFTISRDNAKNTLFLQMTSLRSEDTAMYYCAR
RGLLLDYWGQGTTLTVSS.

In some embodiments, a 3E10 heavy chain is expressed as

MGWSCIILFLVATATGVHSEVQLVESGGGLVKPGGSRKLSCAASGFTFS
NYGMHWVRQAPEKGLEWVAYISSGSSTIYYADTVKGRFTISRDNAKNTL
FLQMTSLRSEDTAMYYCARRGLLLDYWGQGTTLTVSAASTKGPSVFPLA
PSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK (3E10 D31N Variant Heavy
Chain; SEQ ID NO: 13).

In some embodiments, the C-terminal serine of SEQ ID NOS: 1 or 2 is absent or substituted, with, for example, an alanine, in 3E10 heavy chain variable region.

The complementarity determining regions (CDRs) as identified by Kabat are shown with underlining above and include CDR H1.1 (original sequence): DYGMH (SEQ ID NO:15); CDR H1.2 (with D31N mutation): NYGMH (SEQ ID NO:16); CDR H2.1: YISSGSSTIYYADTVKG (SEQ ID NO: 17); CDR H3.1: RGLLLDY (SEQ ID NO:18).

A variant of Kabat CDR H2.1 is YISSGSSTIYYADSVKG (SEQ ID NO: 19).

Additionally, or alternatively, the heavy chain complementarity determining regions (CDRs) can be defined according to the IMGT system. The complementarity determining regions (CDRs) as identified by the IMGT system include CDR H1.3 (original sequence): GFTFSDYG (SEQ ID NO: 20); CDR H1.4 (with D31N mutation): GFTFSNYG (SEQ ID NO:21); CDR H2.2: ISSGSSTI (SEQ ID NO:22); CDR H3.2: ARRGLLLDY (SEQ ID NO: 23).

### 2. 3E10 Light Chain

In some embodiments, a light chain variable region of 3E10 is:

(SEQ ID NO: 7)
DIVLTQSPASLAVSLGQRATISCRASKSVSTSSYSYMHWYQQKPGQPPK
LLIKYASYLESGVPARFSGSGSGTDFTLNIHPVEEEDAATYYCQHSREF
PWTFGGGTKLEIK.

An amino acid sequence for the light chain variable region of 3E10 can also be:

(SEQ ID NO: 8)
DIVLTQSPASLAVSLGQRATISCRASKSVSTSSYSYMHWYQQKPGQPPK
LLIKYASYLESGVPARFSGSGSGTDFHLNIHPVEEEDAATYYCQHSREF
PWTFGGGTKLELK.

In some embodiments, a 3E10 light chain is expressed as

(3E10 WT Light Chain; SEQ ID NO: 14)
MGWSCIILFLVATATGVHSDIVLTQSPASLAVSLGQRATISCRASKSVS
TSSYSYMHWYQQKPGQPPKLLIKYASYLESGVPARFSGSGSGTDFTLNI
HPVEEEDAATYYCQHSREFPWTFGGGTKLEIKRTVAAPSVFIFPPSDEQ
LKSGTASVVCLLNNFYPREAKVQWKVDNALQSGNSQESVTEQDSKDSTY
SLSSTLTLSKADYEKHKVYACEVTHQGLSSPVTKSFNRGEC

Other 3E10 light chain sequences are known in the art. See, for example, Zack, et al., J. Immunol., 15; 154 (4): 1987-94 (1995); GenBank: L16981.1-Mouse Ig rearranged L-chain gene, partial cds; GenBank: AAA65681.1-immunoglobulin light chain, partial [Mus musculus]).

The complementarity determining regions (CDRs) as identified by Kabat are shown with underlining, including

CDR L1.1:
(SEQ ID NO: 24)
RASKSVSTSSYSYMH;
CDR L2.1:
(SEQ ID NO: 25)
YASYLES;
CDR L3.1:
(SEQ ID NO: 26)
QHSREFPWT.

A variant of Kabat CDR L1.1 is RASKSVSTSSYSYLA (SEQ ID NO: 27).

A variant of Kabat CDR L2.1 is YASYLQS (SEQ ID NO:28).

Additionally, or alternatively, the heavy chain complementarity determining regions (CDRs) can be defined according to the IMGT system. The complementarity determining regions (CDRs) as identified by the IMGT system include CDR L1.2 KSVSTSSYSY (SEQ ID NO:29); CDR L2.2: YAS (SEQ ID NO:30); CDR L3.2: QHSREFPWT (SEQ ID NO:26).

In some embodiments, the C-terminal end of sequence of SEQ ID NOS: 7 or 8 further includes an arginine in the 3E10 light chain variable region.

### B. Humanized 3E10

In some embodiments, the antibody is a humanized antibody.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

Exemplary 3E10 humanized sequences are discussed in WO 2015/106290 and WO 2016/033324, and provided below.

### 1. Humanized 3E10 Heavy Chain Variable Regions

In some embodiments, a humanized 3E10 heavy chain variable domain includes

(hVH1, SEQ ID NO: 3)
EVQLVQSGGGLIQPGGSLRLSCAASGFTFSNYGMHWVRQAPGKGLEWVS
YISSGSSTIYYADSVKGRFTISRDNSKNTLYLQMNSLRAEDTAVYYCAR
RGLLLDYWGQGTTVTVSS,
or
(hVH2, SEQ ID NO: 4)
EVQLVESGGGLIQPGGSLRLSCAASGFTFSNYGMHWVRQAPGKGLEWVS
YISSGSSTIYYADSVKGRFTISRDNSKNTLYLQMTSLRAEDTAVYYCAR
RGLLLDYWGQGTTLTVSS,
or
(hVH3, SEQ ID NO: 5)
EVQLQESGGGVVQPGGSLRLSCAASGFTFSNYGMHWIRQAPGKGLEWVS
YISSGSSTIYYADSVKGRFTISRDNSKNTLYLQMNSLRSEDTAVYYCAR
RGLLLDYWGQGTLVTVSS
(hVH4, SEQ ID NO: 6)
EVQLVESGGGLVQPGGSLRLSCSASGFTFSNYGMHWVRQAPGKGLEYVS
YISSGSSTIYYADTVKGRFTISRDNSKNTLYLQMSSLRAEDTAVYYCVK
RGLLLDYWGQGTLVTVSS

### 2. Humanized 3E10 Light Chain Variable Regions

In some embodiments, a humanized 3E10 light chain variable domain includes

(hVL1, SEQ ID NO: 9)
DIQMTQSPSSLSASVGDRVTITCRASKSVSTSSYSYLAWYQQKPEKAPK
LLIKYASYLQSGVPSRFSGSGSGTDFTLTISSLQPEDFATYYCQHSREF
PWTFGAGTKLELK,
or
(hVL2, SEQ ID NO: 10)
DIQMTQSPSSLSASVGDRVTISCRASKSVSTSSYSYMHWYQQKPEKAPK
LLIKYASYLQSGVPSRFSGSGSGTDFTLTISSLQPEDVATYYCQHSREF
PWTFGAGTKLELK,
or
(hVL3, SEQ ID NO: 11)
DIVLTQSPASLAVSPGQRATITCRASKSVSTSSYSYMHWYQQKPGQPPK
LLIYYASYLESGVPARFSGSGSGTDFTLTINPVEANDTANYYCQHSREF
PWTFGQGTKVEIK

### C. Fragments, Variants, and Fusion Proteins

The anti-DNA antibody can be composed of an antibody fragment or fusion protein including an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, at least 99%, or 100% identical to the amino acid sequence of the variable heavy chain and/or light chain of 3E10 or a humanized form thereof (e.g., any of SEQ ID NOS: 1-11, or the heavy and/or light chains of any of SEQ ID NOS: 12-14).

The anti-DNA antibody can be composed of an antibody fragment or fusion protein that includes one or more CDR(s) that is at least 45%, at least 50%, at least 55%, at least 60%, at least 65%, at least 70%, at least 75%, at least 80%, at least 85%, at least 90%, at least 95%, at least 99%, or 100% identical to the amino acid sequence of the CDR(s) of 3E10, or a variant or humanized form thereof (e.g., CDR(s) of any of SEQ ID NOS: 1-11, or SEQ ID NOS: 12-14, or SEQ ID NOS: 15-30). The determination of percent identity of two amino acid sequences can be determined by BLAST protein comparison. In some embodiments, the antibody includes one, two, three, four, five, or all six of the CDRs of the above-described preferred variable domains.

Preferably, the antibody include one of each of a heavy chain CDR1, CDR2, and CDR3 in combination with one of each of a light chain CDR1, CDR2, and CDR3.

Predicted complementarity determining regions (CDRs) of the light chain variable sequence for 3E10 are provided above. See also GenBank: AAA65681.1-immunoglobulin light chain, partial [Mus musculus] and GenBank: L34051.1-Mouse Ig rearranged kappa-chain mRNA V-region. Predicted complementarity determining regions (CDRs) of the heavy chain variable sequence for 3E10 are provide above. See also, for example, Zack, et al., Immunology and Cell Biology, 72:513-520 (1994), GenBank Accession number AAA65679.1. Zach, et al., J. Immunol. 154 (4), 1987-1994 (1995) and GenBank: L16982.1-Mouse Ig reagrranged H-chain gene, partial cds.

Thus, in some embodiments, the cell-penetrating antibody contains the CDRs, or the entire heavy and light chain variable regions, of SEQ ID NO: 1 or 2, or the heavy chain region of SEQ ID NO: 12 or 13; or a humanized form thereof in combination with SEQ ID NO:7 or 8, or the light chain region of SEQ ID NO: 14; or a humanized form thereof. In some embodiments, the cell-penetrating antibody contains the CDRs, or the entire heavy and light chain variable regions, of SEQ ID NO:3, 4, 5, or 6 in combination with SEQ ID NO:9, 10, or 11.

Also included are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific to an antigenic protein of the present disclosure. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

The anti-DNA antibodies can be modified to improve their therapeutic potential. For example, in some embodiments, the cell-penetrating anti-DNA antibody is conjugated to another antibody specific for a second therapeutic target in the cytoplasm and/or nucleus of a target cell. For example, the cell-penetrating anti-DNA antibody can be a fusion protein containing 3E10 Fv and a single chain variable fragment of a monoclonal antibody that specifically binds the second therapeutic target. In other embodiments, the cell-penetrating anti-DNA antibody is a bispecific antibody having a first heavy chain and a first light chain from 3E10 and a second heavy chain and a second light chain from a monoclonal antibody that specifically binds the second therapeutic target.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies. In some embodiments, the anti-DNA antibody may contain two or more linked single chain variable fragments of 3E10 (e.g., 3E10 di-scFv, 3E10 tri-scFv), or conservative variants thereof. In some embodiments, the anti-DNA antibody is a diabody or triabody (e.g., 3E10 diabody, 3E10 triabody). Sequences for single and two or more linked single chain variable fragments of 3E10 are provided in WO 2017/218825 and WO 2016/033321.

The function of the antibody may be enhanced by coupling the antibody or a fragment thereof with a therapeutic agent. Such coupling of the antibody or fragment with the therapeutic agent can be achieved by making an immunoconjugate or by making a fusion protein, or by linking the antibody or fragment to a nucleic acid such as DNA or RNA (e.g., siRNA), comprising the antibody or antibody fragment and the therapeutic agent.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. The DNA sequence will then be expressed by a cell as a single protein. The protein can be engineered to include the full sequence of both original proteins, or only a portion of either. If the two entities are proteins, often linker (or “spacer”) peptides are also added which make it more likely that the proteins fold independently and behave as expected.

In some embodiments, the cell-penetrating antibody is modified to alter its half-life. In some embodiments, it is desirable to increase the half-life of the antibody so that it is present in the circulation or at the site of treatment for longer periods of time. For example, it may be desirable to maintain titers of the antibody in the circulation or in the location to be treated for extended periods of time. In other embodiments, the half-life of the anti-DNA antibody is decreased to reduce potential side effects. Antibody fragments, such as 3E10Fv may have a shorter half-life than full size antibodies. Other methods of altering half-life are known and can be used in the described methods. For example, antibodies can be engineered with Fc variants that extend half-life, e.g., using Xtend™ antibody half-life prolongation technology (Xencor, Monrovia, CA).

### 1. Linkers

The term “linker” as used herein includes, without limitation, peptide linkers. The peptide linker can be any size provided it does not interfere with the binding of the epitope by the variable regions. In some embodiments, the linker includes one or more glycine and/or serine amino acid residues. Monovalent single-chain antibody variable fragments (scFvs) in which the C-terminus of one variable domain are typically tethered to the N-terminus of the other variable domain via a 15 to 25 amino acid peptide or linker. The linker is chosen to permit the heavy chain and light chain to bind together in their proper conformational orientation. Linkers in diabodies, triabodies, etc., typically include a shorter linker than that of a monovalent scFv as discussed above. Di-, tri-, and other multivalent scFvs typically include three or more linkers. The linkers can be the same, or different, in length and/or amino acid composition. Therefore, the number of linkers, composition of the linker(s), and length of the linker(s) can be determined based on the desired valency of the scFv as is known in the art. The linker(s) can allow for or drive formation of a di-, tri-, and other multivalent scFv.

For example, a linker can include 4-8 amino acids. In a particular embodiment, a linker includes the amino acid sequence GQSSRSS (SEQ ID NO: 31). In another embodiment, a linker includes 15-20 amino acids, for example, 18 amino acids. In a particular embodiment, the linker includes the amino acid sequence GQSSRSSSGGGSSGGGGS (SEQ ID NO:32). Other flexible linkers include, but are not limited to, the amino acid sequences Gly-Ser, Gly-Ser-Gly-Ser (SEQ ID NO:33), Ala-Ser, Gly-Gly-Gly-Ser (SEQ ID NO: 34), (Gly₄-Ser)₂ (SEQ ID NO:35) and (Gly₄-Ser)₄ (SEQ ID NO:36), and (Gly-Gly-Gly-Gly-Ser)₃ (SEQ ID NO:37).

2. Exemplary Anti-DNA scFv Sequences

Exemplary murine 3E10 scFv sequences, including mono-, di-, and tri-scFv are disclosed in WO 2016/033321 and WO 2017/218825 and provided below. Cell-penetrating antibodies for use in the disclosed compositions and methods include exemplary scFv, and fragments and variants thereof.

The amino acid sequence for scFv 3E10 (D31N) is:

(SEQ ID NO: 38)
AGIHDIVLTQSPASLAVSLGQRATISCRASKSVSTSSYSYMHWYQQKPG
QPPKLLIKYASYLESGVPARFSGSGSGTDFTLNIHPVEEEDAATYYCQH
SREFPWTFGGGTKLEIKRADAAPGGGGSGGGGSGGGGSEVQLVESGGGL
VKPGGSRKLSCAASGFTFSNYGMHWVRQAPEKGLEWVAYISSGSSTIYY
ADTVKGRFTISRDNAKNTLFLQMTSLRSEDTAMYYCARRGLLLDYWGQG
TTLTVSSLEQKLISEEDLNSAVDHHHHHH.

Annotation of scFy Protein Domains with Reference to SEQ ID NO: 38
AGIH sequence increases solubility (amino acids 1-4 of SEQ ID NO: 38)
Vk variable region (amino acids 5-115 of SEQ ID NO:38)
Initial (6 aa) of light chain CHI (amino acids 116-121 of SEQ ID NO: 38)
(GGGGS)₃ (SEQ ID NO:37) linker (amino acids 122-136 of SEQ ID NO: 38)
VH variable region (amino acids 137-252 of SEQ ID NO:38)
Myc tag (amino acids 253-268 SEQ ID NO:38)
His 6 tag (amino acids 269-274 of SEQ ID NO:38)

### Amino Acid Sequence of 3E10 Di-scFv (D31N)

Di-scFv 3E10 (D31N) is a di-single chain variable fragment including 2× the heavy chain and light chain variable regions of 3E10 and wherein the aspartic acid at position 31 of the heavy chain is mutated to an asparagine. The amino acid sequence for di-scFv 3E10 (D31N) is:

(SEQ ID NO: 39)
AGIHDIVLTQSPASLAVSLGQRATISCRASKSVSTSSYSYMHWYQQKPG
QPPKLLIKYASYLESGVPARFSGSGSGTDFTLNIHPVEEEDAATYYCQH
SREFPWTFGGGTKLEIKRADAAPGGGGSGGGGSGGGGSEVQLVESGGGL
VKPGGSRKLSCAASGFTFSNYGMHWVRQAPEKGLEWVAYISSGSSTIYY
ADTVKGRFTISRDNAKNTLFLQMTSLRSEDTAMYYCARRGLLLDYWGQG
TTLTVSSASTKGPSVFPLAPLESSGSDIVLTQSPASLAVSLGQRATISC
RASKSVSTSSYSYMHWYQQKPGQPPKLLIKYASYLESGVPARFSGSGSG
TDFTLNIHPVEEEDAATYYCQHSREFPWTFGGGTKLEIKRADAAPGGGG
SGGGGSGGGGSEVQLVESGGGLVKPGGSRKLSCAASGFTFSNYGMHWVR
QAPEKGLEWVAYISSGSSTIYYADTVKGRFTISRDNAKNTLFLQMTSLR
SEDTAMYYCARRGLLLDYWGQGTTLTVSSLEQKLISEEDLNSAVDHHHH
HH.

Annotation of Di-scFv Protein Domains with Reference to SEO ID NO: 39
AGIH sequence increases solubility (amino acids 1˜4 of SEQ ID NO: 39)
Vk variable region (amino acids 5-115 of SEQ ID NO:39)
Initial (6 aa) of light chain CHI (amino acids 116-121 of SEQ ID NO: 39)
(GGGGS)₃ (SEQ ID NO:37) linker (amino acids 122-136 of SEQ ID NO: 39)
VH variable region (amino acids 137-252 of SEQ ID NO:39)
Linker between Fv fragments consisting of human IgG CHI initial 13 amino acids (amino acids 253-265 of SEQ ID NO:39)
Swivel sequence (amino acids 266-271 of SEQ ID NO:39) Vk variable region (amino acids 272-382 of SEQ ID NO:39)
Initial (6 aa) of light chain CHI (amino acids 383-388 of SEQ ID NO: 39)
(GGGGS)₃ (SEQ ID NO:37) linker (amino acids 389-403 of SEQ ID NO: 39)
VH variable region (amino acids 404-519 of SEQ ID NO:39)
Myc tag (amino acids 520-535 of SEQ ID NO:39) His 6 tag (amino acids 536-541 of SEQ ID NO:39)

### Amino Acid Sequence for Tri-scFv

Tri-scFv 3E10 (D31N) is a tri-single chain variable fragment including 3× the heavy chain and light chain variable regions of 310E and wherein the aspartic acid at position 31 of the heavy chain is mutated to an asparagine. The amino acid sequence for tri-scFv 3E10 (D31N) is:

(SEQ ID NO: 40)
AGIHDIVLTQSPASLAVSLGQRATISCRASKSVSTSSYSYMHWYQQKPG
QPPKLLIKYASYLESGVPARFSGSGSGTDFTLNIHPVEEEDAATYYCQH
SREFPWTFGGGTKLEIKRADAAPGGGGSGGGGSGGGGSEVQLVESGGGL
VKPGGSRKLSCAASGFTFSNYGMHWVRQAPEKGLEWVAYISSGSSTIYY
ADTVKGRFTISRDNAKNTLFLQMTSLRSEDTAMYYCARRGLLLDYWGQG
TTLTVSSASTKGPSVFPLAPLESSGSDIVLTQSPASLAVSLGQRATISC
RASKSVSTSSYSYMHWYQQKPGQPPKLLIKYASYLESGVPARFSGSGSG
TDFTLNIHPVEEEDAATYYCQHSREFPWTFGGGTKLEIKRADAAPGGGG
SGGGGSGGGGSEVQLVESGGGLVKPGGSRKLSCAASGFTFSNYGMHWVR
QAPEKGLEWVAYISSGSSTIYYADTVKGRFTISRDNAKNTLFLQMTSLR
SEDTAMYYCARRGLLLDYWGQGTTLTVSSASTKGPSVFPLAPLESSGSD
IVLTQSPASLAVSLGQRATISCRASKSVSTSSYSYMHWYQQKPGQPPKL
LIKYASYLESGVPARFSGSGSGTDFTLNIHPVEEEDAATYYCQHSREFP
WTFGGGTKLEIKRADAAPGGGGSGGGGSGGGGSEVQLVESGGGLVKPGG
SRKLSCAASGFTFSNYGMHWVRQAPEKGLEWVAYISSGSSTIYYADTVK
GRFTISRDNAKNTLFLQMTSLRSEDTAMYYCARRGLLLDYWGQGTTLTV
SSLEQKLISEEDLNSAVDHHHHHH.

Annotation of Tri-scFv Protein Domains with Reference to SEO ID NO: 40
AGIH sequence increases solubility (amino acids 1˜4 of SEQ ID NO: 40)
Vk variable region (amino acids 5-115 of SEQ ID NO:40)
Initial (6 aa) of light chain CHI (amino acids 116-121 of SEQ ID NO: 40)
(GGGGS)₃ (SEQ ID NO:37) linker (amino acids 122-136 of SEQ ID NO: 40)
VH variable region (amino acids 137-252 of SEQ ID NO:40)
Linker between Fv fragments consisting of human IgG CHI initial 13 amino acids (amino acids 253-265 of SEQ ID NO:40)
Swivel sequence (amino acids 266-271 of SEQ ID NO:40)
Vk variable region (amino acids 272-382 of SEQ ID NO:40)
Initial (6 aa) of light chain CHI (amino acids 383-388 of SEQ ID NO: 40)
(GGGGS)₃ (SEQ ID NO:37) linker (amino acids 389-403 of SEQ ID NO: 40)
VH variable region (amino acids 404-519 of SEQ ID NO:40)
Linker between Fv fragments consisting of human IgG C_{H}1 initial 13 amino acids (amino acids 520-532 of SEQ ID NO:40)
Swivel sequence (amino acids 533-538 of SEQ ID NO:40)
Vk variable region (amino acids 539-649 of SEQ ID NO:40)
Initial (6 aa) of light chain CHI (amino acids 650-655 of SEQ ID NO: 40)
(GGGGS)₃ (SEQ ID NO:37) linker (amino acids 656-670 of SEQ ID NO: 40)
VH variable region (amino acids 671-786 of SEQ ID NO:40)
Myc tag (amino acids 787-802 of SEQ ID NO:40)
His 6 tag (amino acids 803-808 of SEQ ID NO:40)

WO 2016/033321 and Noble, et al., Cancer Research, 75 (11): 2285-2291 (2015), show that di-scFv and tri-scFv have some improved and additional activities compared to their monovalent counterpart. The subsequences corresponding to the different domains of each of the exemplary fusion proteins are also provided above. One of skill in the art will appreciate that the exemplary fusion proteins, or domains thereof, can be utilized to construct fusion proteins discussed in more detail above. For example, in some embodiments, the di-scFv includes a first scFv including a Vk variable region (e.g., amino acids 5-115 of SEQ ID NO:39, or a functional variant or fragment thereof), linked to a VH variable domain (e.g., amino acids 137-252 of SEQ ID NO:39, or a functional variant or fragment thereof), linked to a second scFv including a Vk variable region (e.g., amino acids 272-382 of SEQ ID NO:39, or a functional variant or fragment thereof), linked to a VH variable domain (e.g., amino acids 404-519 of SEQ ID NO: 39, or a functional variant or fragment thereof). In some embodiments, a tri-scFv includes a di-scFv linked to a third scFv domain including a Vk variable region (e.g., amino acids 539-649 of SEQ ID NO:40, or a functional variant or fragment thereof), linked to a VH variable domain (e.g., amino acids 671-786 of SEQ ID NO:40, or a functional variant or fragment thereof).

The Vk variable regions can be linked to VH variable domains by, for example, a linker (e.g., (GGGGS)₃ (SEQ ID NO:37), alone or in combination with a (6 aa) of light chain CHI (amino acids 116-121 of SEQ ID NO: 39). Other suitable linkers are discussed above and known in the art. scFv can be linked by a linker (e.g., human IgG CHI initial 13 amino acids (253-265) of SEQ ID NO:39), alone or in combination with a swivel sequence (e.g., amino acids 266-271 of SEQ ID NO:39). Other suitable linkers are discussed above and known in the art.

Therefore, a di-scFv can include amino acids 5-519 of SEQ ID NO: 39. A tri-scFv can include amino acids 5-786 of SEQ ID NO:40. In some embodiments, the fusion proteins include additional domains. For example, in some embodiments, the fusion proteins include sequences that enhance solubility (e.g., amino acids 1-4 of SEQ ID NO:39). Therefore, in some embodiments, a di-scFv can include amino acids 1-519 of SEQ ID NO: 39. A tri-scFv can include amino acids 1-786 of SEQ ID NO:40. In some embodiments that fusion proteins include one or more domains that enhance purification, isolation, capture, identification, separation, etc., of the fusion protein. Exemplary domains include, for example, Myc tag (e.g., amino acids 520-535 of SEQ ID NO:39) and/or a His tag (e.g., amino acids 536-541 of SEQ ID NO:39). Therefore, in some embodiments, a di-scFv can include the amino acid sequence of SEQ ID NO:39. A tri-scFv can include the amino acid sequence of SEQ ID NO:40. Other substitutable domains and additional domains are discussed in more detail above.

An exemplary 3E10 humanized Fv sequence is discussed in WO 2016/033324:

(SEQ ID NO: 41)
DIVLTQSPASLAVSPGQRATITCRASKSVSTSSYSYMHWYQQKPGQPPK
LLTYYASYLESGVPARFSGSGSGTDFTLTINPVEANDTANYYCQHSREF
PWTFGQGTKVEIKGGGGSGGGGSGGGGSEVQLVESGGGLVQPGGSLRLS
CSASGFTFSNYGMHWVRQAPGKGLEYVSYISSGSSTIYYADTVKGRFTI
SRDNSKNTLYLQMSSLRAEDTAVYYCVKRGLLLDYWGQGTLVTVSS.

### III. Gene Editing Technology

Gene editing technologies are preferably used in combination with a potentiating agent. Exemplary gene editing technologies include, but are not limited to, triplex-forming oligonucleotides, pseudocomplementary oligonucleotides, CRISPR/Cas, zinc finger nucleases, and TALENs, each of which are discussed in more detail below. As discussed in more detail below, the gene editing technologies may be used in combination with a donor oligonucleotide.

### A. Triplex-Forming Molecules (TFMs)

### 1. Compositions

Compositions containing “triplex-forming molecules,” that bind to duplex DNA in a sequence-specific manner to form a triple-stranded structure include, but are not limited to, triplex-forming oligonucleotides (TFOs), peptide nucleic acids (PNA), and “tail clamp” PNA (tcPNA) are provided. The triplex-forming molecules can be used to induce site-specific homologous recombination in mammalian cells when combined with donor DNA molecules. The donor DNA molecules can contain mutated nucleic acids relative to the target DNA sequence. This is useful to activate, inactivate, or otherwise alter the function of a polypeptide or protein encoded by the targeted duplex DNA. Triplex-forming molecules include triplex-forming oligonucleotides and peptide nucleic acids (PNAs). Triplex-forming molecules are described in U.S. Pat. Nos. 5,962,426, 6,303,376, 7,078,389, 7,279,463, 8,658,608, U.S. Published Application Nos. 2003/0148352, 2010/0172882, 2011/0268810, 2011/0262406, 2011/0293585, and published PCT application numbers WO 1995/001364, WO 1996/040898, WO 1996/039195, WO 2003/052071, WO 2008/086529, WO 2010/123983, WO 2011/053989, WO 2011/133802, WO 2011/13380, Rogers, et al., Proc Natl Acad Sci USA, 99:16695-16700 (2002), Majumdar, et al., Nature Genetics, 20:212-214 (1998), Chin, et al., Proc Natl Acad Sci USA, 105:13514-13519 (2008), and Schleifman, et al., Chem Biol., 18:1189-1198 (2011). As discussed in more detail below, triplex-forming molecules are typically single-stranded oligonucleotides that bind to polypyrimidine: polypurine target motif in a double stranded nucleic acid molecule to form a triple-stranded nucleic acid molecule. The single-stranded oligonucleotide/oligomer typically includes a sequence substantially complementary to the polypurine strand of the polypyrimidine: polypurine target motif via Hoogsteen or reverse Hoogsteen binding.

a. Triplex-Forming Oligonucleotides (TFOs)

Triplex-forming oligonucleotides (TFOs) are defined as oligonucleotides which bind as third strands to duplex DNA in a sequence specific manner. The oligonucleotides are synthetic or isolated nucleic acid molecules which selectively bind to or hybridize with a predetermined target sequence, target region, or target site within or adjacent to a human gene so as to form a triple-stranded structure.

Preferably, the oligonucleotide is a single-stranded nucleic acid molecule between 7 and 40 nucleotides in length, most preferably 10 to 20 nucleotides in length for in vitro mutagenesis and 20 to 30 nucleotides in length for in vivo mutagenesis. The nucleobase (sometimes referred to herein simply as “base”) composition may be homopurine or homopyrimidine. Alternatively, the nucleobase composition may be polypurine or polypyrimidine. However, other compositions are also useful.

The oligonucleotides are preferably generated using known DNA synthesis procedures. In one embodiment, oligonucleotides are generated synthetically. Oligonucleotides can also be chemically modified using standard methods that are well known in the art.

The nucleobase sequence of the oligonucleotides/oligomer is selected based on the sequence of the target sequence, the physical constraints imposed by the need to achieve binding of the oligonucleotide/oligomer within the major groove of the target region, and the need to have a low dissociation constant (K_{d}) for the oligo/target sequence complex. The oligonucleotides/oligomers have a nucleobase composition which is conducive to triple-helix formation and is generated based on one of the known structural motifs for third strand binding (e.g. Hoogsteen binding). The most stable complexes are formed on polypurine:polypyrimidine elements, which are relatively abundant in mammalian genomes. Triplex formation by TFOs can occur with the third strand oriented either parallel or anti-parallel to the purine strand of the nucleic acid duplex. In the anti-parallel, purine motif, the triplets are G.G:C and A.A:T, whereas in the parallel pyrimidine motif, the canonical triplets are C′.G:C and T.A:T. The triplex structures can be stabilized by one, two or three Hoogsteen hydrogen bonds (depending on the nucleobase) between the bases in the TFO strand and the purine strand in the duplex. A review of base compositions and binding properties for third strand binding oligonucleotides and/or peptide nucleic acids is provided in, for example, U.S. Pat. No. 5,422,251, Bentin et al., Nucl. Acids Res., 34 (20): 5790-5799 (2006), and Hansen et al., Nucl. Acids Res., 37 (13): 4498-4507 (2009).

Preferably, the oligonucleotide/oligomer binds to or hybridizes to the target sequence under conditions of high stringency and specificity. Most preferably, the oligonucleotides/oligomers bind in a sequence-specific manner within the major groove of duplex DNA. Reaction conditions for in vitro triple helix formation of an oligonucleotide/oligomer to a double stranded nucleic acid sequence vary from oligo to oligo, depending on factors such as polymer length, the number of G:C and A:T base pairs, and the composition of the buffer utilized in the hybridization reaction. An oligonucleotide substantially complementary, based on the third strand binding code, to the target region of the double-stranded nucleic acid molecule is preferred.

As used herein, a triplex forming molecule is said to be substantially complementary to a target region when the oligonucleotide has a nucleobase composition which allows for the formation of a triple-helix with the target region. As such, an oligonucleotide/oligomer can be substantially complementary to a target region even when there are non-complementary bases present in the oligonucleotide/oligomer. As stated above, there are a variety of structural motifs available which can be used to determine the nucleobase sequence of a substantially complementary oligonucleotide/oligomer

b. Peptide Nucleic Acids (PNA)

In another embodiment, the triplex-forming molecules are peptide nucleic acids (PNAs). Peptide nucleic acids can be considered polymeric molecules in which the sugar phosphate backbone of an oligonucleotide has been replaced in its entirety by repeating substituted or unsubstituted N-(2-aminoethyl)-glycine residues that are linked by amide bonds. The various nucleobases are linked to the backbone by methylene carbonyl linkages. PNAs maintain spacing of the nucleobases in a manner that is similar to that of an oligonucleotide (DNA or RNA), but because the sugar phosphate backbone has been replaced, classic (unsubstituted) PNAs are achiral and neutrally charged molecules. Peptide nucleic acids are composed of peptide nucleic acid residues (sometimes referred to as ‘residues’). The nucleobases can be any of the standard bases (uracil, thymine, cytosine, adenine and guanine) or any of the modified heterocyclic nucleobases described below.

PNAs can bind to DNA via Watson-Crick hydrogen bonds, but with binding affinities significantly higher than those of a corresponding nucleotide composed of DNA or RNA. The neutral backbone of PNAs decreases electrostatic repulsion between the PNA and target DNA phosphates. Under in vitro or in vivo conditions that promote opening of the duplex DNA, PNAs can mediate strand invasion of duplex DNA resulting in displacement of one DNA strand to form a D-loop.

Highly stable triplex PNA:DNA:PNA structures can be formed from a homopurine DNA strand and two PNA strands. The two PNA strands may be two separate PNA molecules (see Bentin et al., Nucl. Acids Res., 34 (20): 5790-5799 (2006) and Hansen et al., Nucl. Acids Res., 37 (13): 4498-4507 (2009)), or two PNA molecules linked together by a linker of sufficient flexibility to form a single bis-PNA molecule (See: U.S. Pat. No. 6,441,130). In both cases, the PNA molecule(s) forms a triplex “clamp” with one of the strands of the target duplex while displacing the other strand of the duplex target. In this structure, one strand forms Watson-Crick base pairs with the DNA strand in the anti-parallel orientation (the Watson-Crick binding portion), whereas the other strand forms Hoogsteen base pairs to the DNA strand in the parallel orientation (the Hoogsteen binding portion). A homopurine strand allows formation of a stable PNA/DNA/PNA triplex. PNA clamps can form at shorter homopurine sequences than those required by triplex-forming oligonucleotides (TFOs) and also do so with greater stability.

Suitable molecules for use in linkers of bis-PNA molecules include, but are not limited to, 8-amino-3,6-dioxaoctanoic acid, referred to as an O-linker, and 6-aminohexanoic acid. Poly(ethylene) glycol monomers can also be used in bis-PNA linkers. A bis-PNA linker can contain multiple linker residues in any combination of two or more of the foregoing. In some embodiments, the PNA oligomers are linked by three 8-amino-2, 6, 10-trioxaoctanoic acid, three 8-amino-3,6-dioxaoctanoic acid, or three 6-aminohexanoic acid molecules.

PNAs can also include other positively charged moieties to increase the solubility of the PNA and increase the affinity of the PNA for duplex DNA. Commonly used positively charged moieties include the amino acids lysine and arginine (e.g., as additional substituents attached to the C- or N-terminus of the PNA oligomer (or a segment thereof) or as a side-chain modification of the backbone (see Huang et al., Arch. Pharm. Res. 35 (3): 517-522 (2012) and Jain et al., JOC, 79 (20): 9567-9577 (2014)), although other positively charged moieties may also be useful (See for Example: U.S. Pat. No. 6,326,479). In some embodiments, the PNA oligomer can have one or more ‘miniPEG’ side chain modifications of the backbone (see, for example, U.S. Pat. No. 9,193,758 and Sahu et al., JOC, 76:5614-5627 (2011)).

Peptide nucleic acids are unnatural synthetic polyamides, prepared using known methodologies, generally as adapted from peptide synthesis processes.

c. Tail Clamp Peptide Nucleic Acids (tcPNA)

Although polypurine:polypyrimidine stretches do exist in mammalian genomes, it is desirable to target triplex formation in the absence of this requirement. In some embodiments such as PNA, triplex-forming molecules include a “tail” added to the end of the Watson-Crick binding portion. Adding additional nucleobases, known as a “tail” or “tail clamp”, to the Watson-Crick binding portion that bind to the target strand outside the triple helix further reduces the requirement for a polypurine:polypyrimidine stretch and increases the number of potential target sites. The tail is most typically added to the end of the Watson-Crick binding sequence furthest from the linker. This molecule therefore mediates a mode of binding to DNA that encompasses both triplex and duplex formation (Kaihatsu, et al., Biochemistry, 42 (47): 13996-4003 (2003); Bentin, et al., Biochemistry, 42 (47): 13987-95 (2003)). For example, if the triplex-forming molecules are tail clamp PNA (tcPNA), the PNA/DNA/PNA triple helix portion and the PNA/DNA duplex portion both produce displacement of the pyrimidine-rich strand, creating an altered helical structure that strongly provokes the nucleotide excision repair pathway and activating the site for recombination with a donor DNA molecule (Rogers, et al., Proc. Natl. Acad. Sci. U.S.A., 99 (26): 16695-700 (2002)).

Tails added to clamp PNAs (sometimes referred to as bis-PNAs) form tail-clamp PNAs (referred to as tcPNAs) that have been described by Kaihatsu, et al., Biochemistry, 42 (47): 13996-4003 (2003); Bentin, et al., Biochemistry, 42 (47): 13987-95 (2003). tcPNAs are known to bind to DNA more efficiently due to low dissociation constants. The addition of the tail also increases binding specificity and binding stringency of the triplex-forming molecules to the target duplex. It has also been found that the addition of a tail to clamp PNA improves the frequency of recombination of the donor oligonucleotide at the target site compared to PNA without the tail.

In some embodiments a PNA tail clamp system includes one or more the following, preferable in the specified orientation/order:
a positively charged region including one or more positively charged amino acids such as lysine;
a region including a number of PNA subunits with Hoogsteen homology with a target sequence;
a linker;
a region including a number of PNA subunits having Watson Crick homology binding with the target sequence;
a region including a number of PNA subunits having Watson Crick homology binding with a tail target sequence;
a positively charged region including one or more positively charged amino acids subunits, such as lysine.

In some embodiments, one or more PNA monomers of the tail target sequence is modified as disclosed herein.

d. PNA Modifications

PNAs can also include other positively charged moieties to increase the solubility of the PNA and increase the affinity of the PNA for duplex DNA. Commonly used positively charged moieties include the amino acids lysine and arginine, although other positively charged moieties may also be useful. Lysine and arginine residues can be added to a bis-PNA linker or can be added to the carboxy or the N-terminus of a PNA strand. Common modifications to PNA are discussed in Sugiyama and Kittaka, Molecules, 18:287-310 (2013)) and Sahu, et al., J. Org. Chem., 76, 5614-5627 (2011), each of which are specifically incorporated by reference in their entireties, and include, but are not limited to, incorporation of charged amino acid residues, such as lysine at the termini or in the interior part of the oligomer; inclusion of polar groups in the backbone, carboxymethylene bridge, and in the nucleobases; chiral PNAs bearing substituents on the original N-(2-aminoethyl)glycine backbone; replacement of the original aminoethylglycyl backbone skeleton with a negatively-charged scaffold; conjugation of high molecular weight polyethylene glycol (PEG) to one of the termini; fusion of PNA to DNA to generate a chimeric oligomer, redesign of the backbone architecture, conjugation of PNA to DNA or RNA. These modifications improve solubility but often result in reduced binding affinity and/or sequence specificity.

Triplex-forming peptide nucleic acid (PNA) oligomers having a γ (also referred to as “gamma”) modification (also referred to as “substitution”) in one or more PNA residues (also referred to as “subunits”) of the PNA oligomer are also provided.

In some embodiments, the some or all of the PNA residues are modified at the gamma position in the polyamide backbone (γPNAs) as illustrated below (wherein “B” is a nucleobase and “R” is a substitution at the gamma position).

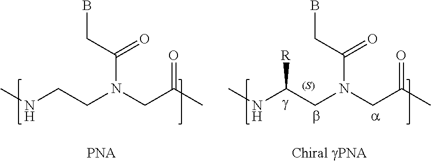

Substitution at the gamma position creates chirality and provides helical pre-organization to the PNA oligomer, yielding substantially increased binding affinity to the target DNA (Rapireddy, et al., Biochemistry, 50 (19): 3913-8 (2011), He et al., “The Structure of a γ-modified peptide nucleic acid duplex”, Mol. BioSyst. 6:1619-1629 (2010); and Sahu et al., “Synthesis and Characterization of Conformationally Preorganized, (R)-Diethylene Glycol-Containing γ-Peptide Nucleic Acids with Superior Hybridization Properties and Water Solubility”, J. Org. Chem, 76:5614-5627) (2011)). Other advantageous properties can be conferred depending on the chemical nature of the specific substitution at the gamma position (the “R” group in the illustration of the Chiral γPNA, above).

One class of γ substitution, is miniPEG, but other residues and side chains can be considered, and even mixed substitutions can be used to tune the properties of the oligomers. “MiniPEG” and “MP” refers to diethylene glycol. MiniPEG-containing γPNAs are conformationally preorganized PNAs that exhibit superior hybridization properties and water solubility as compared to the original PNA design and other chiral γPNAs. Sahu et al., describes γPNAs prepared from L-amino acids that adopt a right-handed helix, and γPNAs prepared from D-amino acids that adopt a left-handed helix. Only the right-handed helical γPNAs hybridize to DNA or RNA with high affinity and sequence selectivity. In the most preferred embodiments, some or all of the PNA residues are miniPEG-containing γPNAs (Sahu, et al., J. Org. Chem., 76, 5614-5627 (2011). In some embodiments, tcPNAs are prepared wherein every other PNA residue on the Watson-Crick binding side of the linker is a miniPEG-containing γPNA. Accordingly, for these embodiments, the tail clamp side of the PNA has alternating classic PNA and miniPEG-containing γPNA residues.

In some embodiments PNA-mediated gene editing are achieved via additional or alternative γ substitutions or other PNA chemical modifications including but limited to those introduced above and below. Examples of γ substitution with other side chains include that of alanine, serine, threonine, cysteine, valine, leucine, isoleucine, methionine, proline, phenylalanine, tyrosine, aspartic acid, glutamic acid, asparagine, glutamine, histidine, lysine, arginine, and the derivatives thereof. The “derivatives thereof” herein are defined as those chemical moieties that are covalently attached to these amino acid side chains, for instance, to that of serine, cysteine, threonine, tyrosine, aspartic acid, glutamic acid, asparagine, glutamine, lysine, and arginine.

In addition to γPNAs showing consistently improved gene editing potency the level of off-target effects in the genome remains extremely low. This is in keeping with the lack of any intrinsic nuclease activity in the PNAs (in contrast to ZFNs or CRISPR/Cas9 or TALENS), and reflects the mechanism of triplex-induced gene editing, which acts by creating an altered helix at the target-binding site that engages endogenous high fidelity DNA repair pathways. As discussed above, the SCF/c-Kit pathway also stimulates these same pathways, providing for enhanced gene editing without increasing off-target risk or cellular toxicity.

Additionally, any of the triplex forming sequences can be modified to include guanidine-G-clamp (“G-clamp”) PNA residues(s) to enhance PNA binding, wherein the G-clamp is linked to the backbone as any other nucleobase would be. γPNAs with substitution of cytosine by G-clamp (9-(2-guanidinoethoxy) phenoxazine), a cytosine analog that can form five H-bonds with guanine, and can also provide extra base stacking due to the expanded phenoxazine ring system and substantially increased binding affinity. In vitro studies indicate that a single G-clamp substitution for C can substantially enhance the binding of a PNA-DNA duplex by 230C (Kuhn, et al., Artificial DNA, PNA & XNA, 1 (1): 45-53 (2010)). As a result, γPNAs containing G-clamp substitutions can have further increased activity.

The structure of a G-clamp monomer-to-G base pair (G-clamp indicated by the “X”) is illustrated below in comparison to C-G base pair.

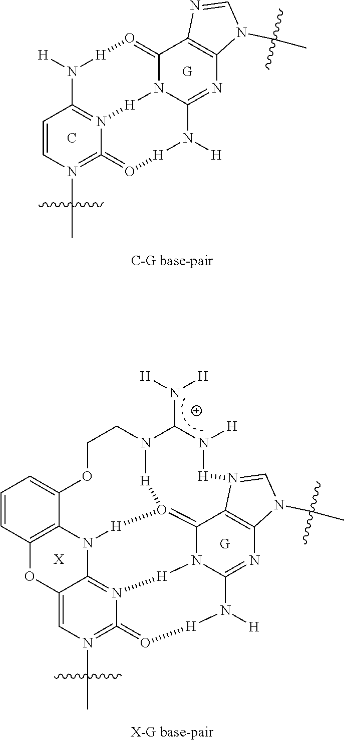

Some studies have shown improvements using D-amino acids in peptide synthesis.

In particular embodiments, the gene editing composition includes at least one peptide nucleic acid (PNA) oligomer. The at least one PNA oligomer can be a modified PNA oligomer including at least one modification at a gamma position of a backbone carbon. The modified PNA oligomer can include at least one miniPEG modification at a gamma position of a backbone carbon. The gene editing composition can include at least one donor oligonucleotide. The gene editing composition can modify a target sequence within a fetal genome.

The PNA can include a Hoogsteen binding peptide nucleic acid (PNA) segment and a Watson-Crick binding PNA segment collectively totaling no more than 50 nucleobases in length, wherein the two segments bind or hybridize to a target region of a genomic DNA comprising a polypurine stretch to induce strand invasion, displacement, and formation of a triple-stranded composition among the two PNA segments and the polypurine stretch of the genomic DNA, wherein the Hoogsteen binding segment binds to the target region by Hoogsteen binding for a length of least five nucleobases, and wherein the Watson-Crick binding segment binds to the target region by Watson-Crick binding for a length of least five nucleobases.

The PNA segments can include a gamma modification of a backbone carbon. The gamma modification can be a gamma miniPEG modification. The Hoogsteen binding segment can include one or more chemically modified cytosines selected from the group consisting of pseudocytosine, pseudoisocytosine, and 5-methylcytosine. The Watson-Crick binding segment can include a sequence of up to fifteen nucleobases that binds to the target duplex by Watson-Crick binding outside of the triplex. The two segments can be linked by a linker. In some embodiments, all of the peptide nucleic acid residues in the Hoogsteen-binding segment only, in the Watson-Crick-binding segment only, or across the entire PNA oligomer include a gamma modification of a backbone carbon. In some embodiments, one or more of the peptide nucleic acid residues in the Hoogsteen-binding segment only or in the Watson-Crick-binding segment only of the PNA oligomer include a gamma modification of a backbone carbon. In some embodiments, alternating peptide nucleic acid residues in the Hoogsteen-binding portion only, in the Watson-Crick-binding portion only, or across the entire PNA oligomer include a gamma modification of a backbone carbon.

In some embodiments, least one gamma modification of the backbone carbon is a gamma miniPEG modification. In some embodiments, at least one gamma modification is a side chain of an amino acid selected from the group consisting of alanine, serine, threonine, cysteine, valine, leucine, isoleucine, methionine, phenylalanine, tyrosine, aspartic acid, glutamic acid, asparagine, glutamine, histidine, lysine, arginine, and the derivatives thereof. In some embodiments, all gamma modifications are gamma miniPEG modifications. Optionally, at least one PNA segment comprises a G-clamp (9-(2-guanidinoethoxy) phenoxazine).

### 2. Triplex-Forming Target Sequence Considerations

The triplex-forming molecules bind to a predetermined target region referred to herein as the “target sequence,” “target region,” or “target site.” The target sequence for the triplex-forming molecules can be within or adjacent to a human gene encoding, for example the beta globin, cystic fibrosis transmembrane conductance regulator (CFTR) or other gene discussed in more detail below, or an enzyme necessary for the metabolism of lipids, glycoproteins, or mucopolysaccharides, or another gene in need of correction. The target sequence can be within the coding DNA sequence of the gene or within an intron. The target sequence can also be within DNA sequences which regulate expression of the target gene, including promoter or enhancer sequences or sites that regulate RNA splicing.

The nucleotide sequences of the triplex-forming molecules are selected based on the sequence of the target sequence, the physical constraints, and the preference for a low dissociation constant (K_{d}) for the triplex-forming molecules/target sequence. As used herein, triplex-forming molecules are said to be substantially complementary to a target region when the triplex-forming molecules has a nucleobase composition which allows for the formation of a triple-helix with the target region. A triplex-forming molecule can be substantially complementary to a target region even when there are non-complementary nucleobases present in the triplex-forming molecules.

There are a variety of structural motifs available which can be used to determine the nucleotide sequence of a substantially complementary oligonucleotide. Preferably, the triplex-forming molecules bind to or hybridize to the target sequence under conditions of high stringency and specificity. Reaction conditions for in vitro triple helix formation of an triplex-forming molecules probe or primer to a nucleic acid sequence vary from triplex-forming molecules to triplex-forming molecules, depending on factors such as the length triplex-forming molecules, the number of G:C and A:T base pairs, and the composition of the buffer utilized in the hybridization reaction.

a. Target Sequence Considerations for TFOs

Preferably, the TFO is a single-stranded nucleic acid molecule between 7 and 40 nucleotides in length, most preferably 10 to 20 nucleotides in length for in vitro mutagenesis and 20 to 30 nucleotides in length for in vivo mutagenesis. The base composition may be homopurine or homopyrimidine. Alternatively, the base composition may be polypurine or polypyrimidine. However, other compositions are also useful. Most preferably, the oligonucleotides bind in a sequence-specific manner within the major groove of duplex DNA. An oligonucleotide substantially complementary, based on the third strand binding code, to the target region of the double-stranded nucleic acid molecule is preferred. The oligonucleotides will have a base composition which is conducive to triple-helix formation and will be generated based on one of the known structural motifs for third strand binding. The most stable complexes are formed on polypurine:polypyrimidine elements, which are relatively abundant in mammalian genomes. Triplex formation by TFOs can occur with the third strand oriented either parallel or anti-parallel to the purine strand of the duplex. In the anti-parallel, purine motif, the triplets are G.G:C and A.A:T, whereas in the parallel pyrimidine motif, the canonical triplets are C⁺.G:C and T.A:T. The triplex structures are stabilized by two Hoogsteen hydrogen bonds between the bases in the TFO strand and the purine strand in the duplex. A review of base compositions for third strand binding oligonucleotides is provided in U.S. Pat. No. 5,422,251.

TFOs are preferably generated using known DNA and/or PNA synthesis procedures. In one embodiment, oligonucleotides are generated synthetically. Oligonucleotides can also be chemically modified using standard methods that are well known in the art.

b. Target Sequence Considerations for PNAs

Some triplex-forming molecules, such as PNA, PNA clamps and tail clamp PNAs (tcPNAs) invade the target duplex, with displacement of the polypyrimidine strand, and induce triplex formation with the polypurine strand of the target duplex by both Watson-Crick and Hoogsteen binding. Preferably, both the Watson-Crick and Hoogsteen binding portions of the triplex-forming molecules are substantially complementary to the target sequence. Although, as with triplex-forming oligonucleotides, a homopurine strand is needed to allow formation of a stable PNA/DNA/PNA triplex, PNA clamps can form at shorter homopurine sequences than those required by triplex-forming oligonucleotides and also do so with greater stability.

Preferably, PNAs are between 6 and 50 nucleobase-containing residues in length. The Watson-Crick portion should be 9 or more nucleobase-containing residues in length, optionally including a tail sequence. More preferably, the Watson-Crick binding portion is between about 9 and 30 nucleobase-containing residues in length, optionally including a tail sequence of between 0 and about 15 nucleobase-containing residues. More preferably, the Watson-Crick binding portion is between about 10 and 25 nucleobase-containing residues in length, optionally including a tail sequence of between 0 and about 10 nucleobase-containing residues in length. In the most preferred embodiment, the Watson-Crick binding portion is between 15 and 25 nucleobase-containing residues in length, optionally including a tail sequence of between 5 and 10 nucleobase-containing residues in length. The Hoogsteen binding portion should be 6 or more nucleobase residues in length. Most preferably, the Hoogsteen binding portion is between about 6 and 15 nucleobase-containing residues in length, inclusive.

The triplex-forming molecules are designed to target the polypurine strand of a polypurine:polypyrimidine stretch in the target duplex nucleotide. Therefore, the base composition of the triplex-forming molecules may be homopyrimidine. Alternatively, the base composition may be polypyrimidine. The addition of a “tail” reduces the requirement for polypurine:polypyrimidine run. Adding additional nucleobase-containing residues, known as a “tail,” to the Watson-Crick binding portion of the triplex-forming molecules allows the Watson-Crick binding portion to bind/hybridize to the target strand outside the site of polypurine sequence for triplex formation. These additional bases further reduce the requirement for the polypurine:polypyrimidine stretch in the target duplex and therefore increase the number of potential target sites. Triplex-forming molecules (TFMs) including, e.g., triplex-forming oligonucleotides (TFOs) and helix-invading peptide nucleic acids (bis-PNAs and tcPNAs), also generally utilize a polypurine:polypyrimidine sequence to a form a triple helix. Traditional nucleic acid TFOs may need a stretch of at least 15 and preferably 30 or more nucleobase-containing residues. Peptide nucleic acids need fewer purines to a form a triple helix, although at least 10 or preferably more may be needed. Peptide nucleic acids including a tail, also referred to tail clamp PNAs, or tcPNAs, require even fewer purines to a form a triple helix. A triple helix may be formed with a target sequence containing fewer than 8 purines. Therefore, PNAs should be designed to target a site on duplex nucleic acid containing between 6-30 polypurine:polypyrimidines, preferably, 6-25 polypurine:polypyrimidines, more preferably 6-20 polypurine:polypyrimidines.

The addition of a “mixed-sequence” tail to the Watson-Crick-binding strand of the triplex-forming molecules such as PNAs also increases the length of the triplex-forming molecule and, correspondingly, the length of the binding site. This increases the target specificity and size of the lesion created at the target site and disrupts the helix in the duplex nucleic acid, while maintaining a low requirement for a stretch of polypurine:polypyrimidines. Increasing the length of the target sequence improves specificity for the target, for example, a target of 17 base pairs will statistically be unique in the human genome. Relative to a smaller lesion, it is likely that a larger triplex lesion with greater disruption of the underlying DNA duplex will be detected and processed more quickly and efficiently by the endogenous DNA repair machinery that facilitates recombination of the donor oligonucleotide.

The triple-forming molecules are preferably generated using known synthesis procedures. In one embodiment, triplex-forming molecules are generated synthetically. Triplex-forming molecules can also be chemically modified using standard methods that are well known in the art.

### B. Pseudocomplementary Oligonucleotides/PNAs

The gene editing technology can be pseudocomplementary oligonucleotides such as those disclosed in U.S. Pat. No. 8,309,356. “Double duplex-forming molecules,” are oligonucleotides that bind to duplex DNA in a sequence-specific manner to form a four-stranded structure. Double duplex-forming molecules, such as a pair of pseudocomplementary oligonucleotides/PNAs, can induce recombination with a donor oligonucleotide at a chromosomal site in mammalian cells. Pseudocomplementary oligonucleotides/PNAs are complementary oligonucleotides/PNAs that contain one or more modifications such that they do not recognize or hybridize to each other, for example due to steric hindrance, but each can recognize and hybridize to its complementary nucleic acid strands at the target site. As used herein the term ‘pseudocomplementary oligonucleotide(s)’ include pseudocomplementary peptide nucleic acids (pcPNAs). A pseudocomplementary oligonucleotide is said to be substantially complementary to a target region when the oligonucleotide has a base composition which allows for the formation of a double duplex with the target region. As such, an oligonucleotide can be substantially complementary to a target region even when there are non-complementary bases present in the pseudocomplementary oligonucleotide.

This strategy can be more efficient and provides increased flexibility over other methods of induced recombination such as triple-helix oligonucleotides and bis-peptide nucleic acids which prefer a polypurine sequence in the target double-stranded DNA. The design ensures that the pseudocomplementary oligonucleotides do not pair with each other but instead bind the cognate nucleic acids at the target site, inducing the formation of a double duplex.

The predetermined region that the double duplex-forming molecules bind to can be referred to as a “double duplex target sequence,” “double duplex target region,” or “double duplex target site.” The double duplex target sequence (DDTS) for the double duplex-forming molecules can be, for example, within or adjacent to a human gene in need of induced gene correction. The DDTS can be within the coding DNA sequence of the gene or within introns. The DDTS can also be within DNA sequences which regulate expression of the target gene, including promoter or enhancer sequences.

The nucleotide/nucleobase sequence of the pseudocomplementary oligonucleotides is selected based on the sequence of the DDTS. Therapeutic administration of pseudocomplementary oligonucleotides involves two single stranded oligonucleotides unlinked, or linked by a linker. One pseudocomplementary oligonucleotide strand is complementary to the DDTS, while the other is complementary to the displaced DNA strand. The use of pseudocomplementary oligonucleotides, particularly pcPNAs are not subject to limitation on sequence choice and/or target length and specificity as are triplex-forming oligonucleotides, helix-invading peptide nucleic acids (bis-PNAs and tcPNAs) and side-by-side minor groove binders. Pseudocomplementary oligonucleotides do not require third-strand Hoogsteen-binding, and therefore are not restricted to homopurine targets. Pseudocomplementary oligonucleotides can be designed for mixed, general sequence recognition of a desired target site. Preferably, the target site contains an A:T base pair content of about 40% or greater. Preferably pseudocomplementary oligonucleotides are between about 8 and 50 nucleobase-containing residues in length, more preferably 8 to 30, even more preferably between about 8 and 20 nucleobase-containing residues in length.

The pseudocomplementary oligonucleotides should be designed to bind to the target site (DDTS) at a distance of between about 1 to 800 bases from the target site of the donor oligonucleotide. More preferably, the pseudocomplementary oligonucleotides bind at a distance of between about 25 and 75 bases from the donor oligonucleotide. Most preferably, the pseudocomplementary oligonucleotides bind at a distance of about 50 bases from the donor oligonucleotide. Preferred pcPNA sequences for targeted repair of a mutation in the β-globin intron IVS2 (G to A) are described in U.S. Pat. No. 8,309,356.

Preferably, the pseudocomplementary oligonucleotides bind/hybridize to the target nucleic acid molecule under conditions of high stringency and specificity. Most preferably, the oligonucleotides bind in a sequence-specific manner and induce the formation of double duplex. Specificity and binding affinity of the pseudocomplemetary oligonucleotides may vary from oligomer to oligomer, depending on factors such as length, the number of G:C and A:T base pairs, and the formulation.

### C. CRISPR/Cas

In some embodiments, the gene editing composition is the CRISPR/Cas system. CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15: 339 (6121): 819-823 (2013) and Jinek, et al., Science, 337 (6096): 816-21 (2012)). By transfecting a cell with the required elements including a cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA: tracrRNA duplex as described in Cong, Science, 15: 339 (6121): 819-823 (2013) and Jinek, et al., Science, 337 (6096): 816-21 (2012)). A single fused crRNA-tracrRNA construct can also be referred to as a guide RNA or gRNA (or single-guide RNA (sgRNA)). Within a sgRNA, the crRNA portion can be identified as the “target sequence” and the tracrRNA is often referred to as the “scaffold.”

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA sequences.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligomers that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

In some embodiments, a vector includes a regulatory element operably linked to an enzyme-coding sequence encoding a CRISPR enzyme, such as a Cas protein. Non-limiting examples of Cas proteins include Casl, CaslB, Cas2, Cas3, Cas4, Cas5, Cas6, Cas7, Cas8, Cas9 (also known as Csnl and Csx12), CasIO, Csy1, Csy2, Csy3, Cse1, Cse2, Cscl, Csc2, Csa5, Csn2, Csm2, Csm3, Csm4, Csm5, Csm6, Cmr1, Cmr3, Cmr4, Cmr5, Cmr6, Csbl, Csb2, Csb3, Csx17, Csx14, CsxIO, Csx16, CsaX, Csx3, Csx1, Csx15, Csfl, Csf2, Csf3, Csf4, Cpf1, homologues thereof, or modified versions thereof. In some embodiments, the unmodified CRISPR enzyme has DNA cleavage activity, such as Cas9. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

The CRISPR/Cas system may contain an enzyme that is mutated with respect to a corresponding wild-type enzyme such that the mutated CRISPR enzyme lacks the ability to cleave one or both strands of a target polynucleotide containing a target sequence. By independently mutating one of the two Cas9 nuclease domains, the Cas9 nickase was developed. For example, an aspartate-to-alanine substitution (D10A) in the RuvC I catalytic domain of Cas9 from S. pyogenes converts Cas9 from a nuclease that cleaves both strands to a nickase (cleaves a single strand). Other residues can be mutated to achieve the above effects (i.e. inactivate one or the other nuclease portions). As non-limiting examples, residues D10, G12, G17, E762, H840, N854, N863, H982, H983, A984, D986, and/or A987 can be substituted. Specific mutations that render Cas9 a nickase include, without limitation, H840A, N854A, and N863A. Mutations other than alanine substitutions are also suitable. Two or more catalytic domains of Cas9 (RuvC I, RuvC II, and RuvC III) can be mutated to produce a mutated Cas9 substantially lacking all DNA cleavage activity. A D10A mutation may be combined with one or more of H840A, N854A, or N863A mutations to produce a Cas9 enzyme substantially lacking all DNA cleavage activity (e.g., when activity of the mutated enzyme is less than about 25%, 10%, 5%>, 1%>, 0.1%>, 0.01%, or lower with respect to its non-mutated form).

Preferably, variants of Cas9, such as for example, a Cas9 nickase are employed in the gene editing technologies containing a CRISPR/Cas system. Nickases can lower the probability of off-target editing, for example, when used with two adjacent gRNAs. A Cas9 nickase having a D10A mutation cleaves only the target strand. Conversely, a Cas9 nickase having an H840A mutation in the HNH domain creates a non-target strand-cleaving nickase. Instead of cutting both strands bluntly with WT Cas9 and one gRNA, one can create a staggered cut using a Cas9 nickase and two gRNAs. This provides even greater control over precise gene integration and insertion. Because both nicking Cas9 enzymes must effectively nick their target DNA, paired nickases have significantly lower off-target effects compared to the double-strand-cleaving Cas9 system, and are generally more effective tools. In a preferred embodiment, the gene editing technology is a Crispr/Cas9 nickase (e.g., D10A, H840A, N854A, and N863A nickase). In a more preferred embodiment, the gene editing technology is a Crispr/Cas9 D10A nickase.

### D. Zinc Finger Nucleases

In some embodiments, the element that induces a single or a double strand break in the target cell's genome is a nucleic acid construct or constructs encoding a zinc finger nucleases (ZFNs). ZFNs are typically fusion proteins that include a DNA-binding domain derived from a zinc-finger protein linked to a cleavage domain.

The most common cleavage domain is the Type IIS enzyme Fokl. Fok1 catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. Proc., Natl. Acad. Sci. USA 89 (1992): 4275-4279; Li et al. Proc. Natl. Acad. Sci. USA, 90:2764-2768 (1993); Kim et al. Proc. Natl. Acad. Sci. USA. 91:883-887 (1994a); Kim et al. J. Biol. Chem. 269:31, 978-31,982 (1994b). One or more of these enzymes (or enzymatically functional fragments thereof) can be used as a source of cleavage domains.

The DNA-binding domain, which can, in principle, be designed to target any genomic location of interest, can be a tandem array of Cys2His₂ zinc fingers, each of which generally recognizes three to four nucleotides in the target DNA sequence. The CyszHisz domain has a general structure: Phe (sometimes Tyr)-Cys-(2 to 4 amino acids)-Cys-(3 amino acids)-Phe (sometimes Tyr)-(5 amino acids)-Leu-(2 amino acids)-His-(3 amino acids)-His. By linking together multiple fingers (the number varies: three to six fingers have been used per monomer in published studies), ZFN pairs can be designed to bind to genomic sequences 18-36 nucleotides long.

Engineering methods include, but are not limited to, rational design and various types of empirical selection methods. Rational design includes, for example, using databases including triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, U.S. Pat. Nos. 6,140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

### E. Transcription Activator-Like Effector Nucleases

In some embodiments, the element that induces a single or a double strand break in the target cell's genome is a nucleic acid construct or constructs encoding a transcription activator-like effector nuclease (TALEN). TALENs have an overall architecture similar to that of ZFNs, with the main difference that the DNA-binding domain comes from TAL effector proteins, transcription factors from plant pathogenic bacteria. The DNA-binding domain of a TALEN is a tandem array of amino acid repeats, each about 34 residues long. The repeats are very similar to each other; typically they differ principally at two positions (amino acids 12 and 13, called the repeat variable diresidue, or RVD). Each RVD specifies preferential binding to one of the four possible nucleotides, meaning that each TALEN repeat binds to a single base pair, though the NN RVD is known to bind adenines in addition to guanine. TAL effector DNA binding is mechanistically less well understood than that of zinc-finger proteins, but their seemingly simpler code could prove very beneficial for engineered-nuclease design. TALENs also cleave as dimers, have relatively long target sequences (the shortest reported so far binds 13 nucleotides per monomer) and appear to have less stringent requirements than ZFNs for the length of the spacer between binding sites. Monomeric and dimeric TALENs can include more than 10, more than 14, more than 20, or more than 24 repeats.

Methods of engineering TAL to bind to specific nucleic acids are described in Cermak, et al, Nucl. Acids Res. 1-11 (2011). U.S. Published Application No. 2011/0145940, which discloses TAL effectors and methods of using them to modify DNA. Miller et al. Nature Biotechnol 29:143 (2011) reported making TALENs for site-specific nuclease architecture by linking TAL truncation variants to the catalytic domain of Fokl nuclease. The resulting TALENs were shown to induce gene modification in immortalized human cells. General design principles for TALEN binding domains can be found in, for example, WO 2011/072246.

### IV. Donor Oligonucleotides

In some embodiments, the gene editing compositions include or are administered in combination with a donor oligonucleotide. The donor oligonucleotide may or may not be not covalently linked to the cell-penetrating antibody used as a potentiating agent. For example, the donor oligonucleotide may form a non-covalent complex with the cell-penetrating antibody. The donor oligonucleotide (e.g., DNA or RNA, or combination thereof) may be single stranded or double stranded. Preferably, the oligonucleotide is single stranded DNA.

Generally, in the case of gene therapy, the donor oligonucleotide includes a sequence that can correct a mutation(s) in the host genome, though in some embodiments, the donor introduces a mutation that can, for example, reduce expression of an oncogene or a receptor that facilitates HIV infection. In addition to containing a sequence designed to introduce the desired correction or mutation, the donor oligonucleotide may also contain synonymous (silent) mutations (e.g., 7 to 10). The additional silent mutations can facilitate detection of the corrected target sequence using allele-specific PCR of genomic DNA isolated from treated cells.

The donor oligonucleotide can exist in single stranded (ss) or double stranded (ds) form (e.g., ssDNA, dsDNA). The donor oligonucleotide can be of any length. For example, the size of the donor oligonucleotide may be between 1 to 800 nucleotides. In one embodiment, the donor oligonucleotide is between 25 and 200 nucleotides. In some embodiments, the donor oligonucleotide is between 100 and 150 nucleotides. In a further embodiment, the donor nucleotide is about 40 to 80 nucleotides in length. The donor oligonucleotide may be about 60 nucleotides in length. ssDNAs of length 25-200 are active. Most studies have been with ssDNAs of length 60-70. Longer ones of length 70-150 also work. The preferred length is 60.

Successful recombination of the donor sequence results in a change of the sequence of the target region. Donor oligonucleotides are also referred to as donor fragments, donor nucleic acids, donor DNA, or donor DNA fragments. It is understood in the art that a greater number of homologous positions within the donor fragment will increase the probability that the donor fragment will be recombined into the target sequence, target region, or target site.

Target sequences can be within the coding DNA sequence of the gene or within introns. Target sequences can also be within DNA sequences which regulate expression of the target gene, including promoter or enhancer sequences or sequences that regulate RNA splicing.

The donor sequence can contain one or more nucleic acid sequence alterations compared to the sequence of the region targeted for recombination, for example, a point mutation, a substitution, a deletion, or an insertion of one or more nucleotides. Deletions and insertions can result in frameshift mutations or deletions. Point mutations can cause missense or nonsense mutations. These mutations may disrupt, reduce, stop, increase, improve, or otherwise alter the expression of the target gene.

The donor oligonucleotide may correspond to the wild type sequence of a gene (or a portion thereof), for example, a mutated gene involved with a disease or disorder (e.g., hemophilia, muscular dystrophy, globinopathies, cystic fibrosis, xeroderma pigmentosum, lysosomal storage diseases, immune deficiency syndromes such as X-linked severe combined immunodeficiency and ADA deficiency, tyrosinemia, Fanconi anemia, the red cell disorder spherocytosis, alpha-1-anti-trypsin deficiency, Wilson's disease, Leber's hereditary optic neuropathy, and chronic granulomatous disorder).

One or more (e.g., 1, 2, 3, 4, 5, 6, 7, 8, 9, 10 or more) different donor oligonucleotide sequences may be used in accordance with the disclosed methods. This may be useful, for example, to create a heterozygous target gene where the two alleles contain different modifications.

Donor oligonucleotides are preferably DNA oligonucleotides, composed of the principal naturally-occurring nucleotides (uracil, thymine, cytosine, adenine and guanine) as the heterocyclic bases, deoxyribose as the sugar moiety, and phosphate ester linkages. Donor oligonucleotides may include modifications to nucleobases, sugar moieties, or backbone/linkages, depending on the desired structure of the replacement sequence at the site of recombination or to provide some resistance to degradation by nucleases. For example, the terminal three inter-nucleoside linkages at each end of a ssDNA oligonucleotide (both 5′ and 3′ ends) may be replaced with phosphorothioate linkages in lieu of the usual phosphodiester linkages, thereby providing increased resistance to exonucleases. Modifications to the donor oligonucleotide should not prevent the donor oligonucleotide from successfully recombining at the recombination target sequence.

Donor oligonucleotides can be either single stranded or double stranded, and can target one or both strands of the genomic sequence at a target locus. The donors are typically presented as single stranded DNA sequences targeting one strand of the target genomic locus. However, even where not expressly provided, the reverse complement of each donor, and double stranded DNA sequences, are also disclosed based on the provided sequences. In some embodiments, the donor oligonucleotide is a functional fragment of the disclosed sequence, or the reverse complement, or double stranded DNA thereof.

In some embodiments, the donor oligonucleotide includes 1, 2, 3, 4, 5, 6, or more optional phosphorothioate internucleoside linkages. In some embodiments, the donor includes phosphorothioate internucleoside linkages between first 2, 3, 4 or 5 nucleotides, and/or the last 2, 3, 4, or 5 nucleotides in the donor oligonucleotide.

### A. Preferred Donor Oligonucleotide Design for

### Triplex and Double-Duplex Based Technologies

The triplex-forming molecules including peptide nucleic acids may be administered in combination with, or tethered to, a donor oligonucleotide via a mixed sequence linker or used in conjunction with a non-tethered donor oligonucleotide that is substantially homologous to the target sequence. Triplex-forming molecules can induce recombination of a donor oligonucleotide sequence up to several hundred base pairs away. It is preferred that the donor oligonucleotide sequence is between 1 to 800 bases from the target binding site of the triplex-forming molecules. More preferably the donor oligonucleotide sequence is between 25 to 75 bases from the target binding site of the triplex-forming molecules. Most preferably that the donor oligonucleotide sequence is about 50 nucleotides from the target binding site of the triplex-forming molecules.

The donor sequence can contain one or more nucleic acid sequence alterations compared to the sequence of the region targeted for recombination, for example, a substitution, a deletion, or an insertion of one or more nucleotides. Successful recombination of the donor sequence results in a change of the sequence of the target region. Donor oligonucleotides are also referred to as donor fragments, donor nucleic acids, donor DNA, or donor DNA fragments. This strategy exploits the ability of a triplex to provoke DNA repair, potentially increasing the probability of recombination with the homologous donor DNA. It is understood in the art that a greater number of homologous positions within the donor fragment will increase the probability that the donor fragment will be recombined into the target sequence, target region, or target site. Tethering of a donor oligonucleotide to a triplex-forming molecule facilitates target site recognition via triple helix formation while at the same time positioning the tethered donor fragment for possible recombination and information transfer. Triplex-forming molecules also effectively induce homologous recombination of non-tethered donor oligonucleotides. The term “recombinagenic” as used herein, is used to define a DNA fragment, oligonucleotide, peptide nucleic acid, or composition as being able to recombine into a target site or sequence or induce recombination of another DNA fragment, oligonucleotide, or composition.

Non-tethered or unlinked fragments may range in length from 20 nucleotides to several thousand. The donor oligonucleotide molecules, whether linked or unlinked, can exist in single stranded or double stranded form. The donor fragment to be recombined can be linked or un-linked to the triplex-forming molecules. The linked donor fragment may range in length from 4 nucleotides to 100 nucleotides, preferably from 4 to 80 nucleotides in length. However, the unlinked donor fragments have a much broader range, from 20 nucleotides to several thousand. In one embodiment the oligonucleotide donor is between 25 and 80 nucleobases. In a further embodiment, the non-tethered donor nucleotide is about 50 to 60 nucleotides in length.

Compositions including triplex-forming molecules such as tcPNA may include one or more than one donor oligonucleotides. More than one donor oligonucleotides may be administered with triplex-forming molecules in a single transfection, or sequential transfections.

### B. Preferred Donor Oligonucleotides Design for Nuclease-Based Technologies

The nuclease activity of the described genome editing systems cleave target DNA to produce single or double strand breaks in the target DNA. Double strand breaks can be repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from a donor polynucleotide to the target DNA. As such, new nucleic acid material can be inserted/copied into the site. The modifications of the target DNA due to NHEJ and/or homology-directed repair can be used to induce gene correction, gene replacement, gene tagging, transgene insertion, nucleotide deletion, gene disruption, gene mutation, etc. It is believed that as a potentiating agent, 3E10 promotes recombination by shifting the balance of DNA repair and recombination pathways from one that is RAD51 mediated to one that is RAD52 mediated.

A polynucleotide including a donor sequence to be inserted at the cleavage site is provided to the cell to be edited. The donor polynucleotide typically contains sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology.

The donor sequence may or may not be identical to the genomic sequence that it replaces. The donor sequence may correspond to the wild type sequence (or a portion thereof) of the target sequence (e.g., a gene). The donor sequence may contain at least one or more single base changes, insertions, deletions, inversions or rearrangements with respect to the genomic sequence, so long as sufficient homology is present to support homology-directed repair. In some embodiments, the donor sequence includes a non-homologous sequence flanked by two regions of homology, such that homology-directed repair between the target DNA region and the two flanking sequences results in insertion of the non-homologous sequence at the target region.

When the genome editing composition includes a donor polynucleotide sequence that includes at least a segment with homology to the target DNA sequence, the methods can be used to add, i.e., insert or replace, nucleic acid material to a target DNA sequence (e.g., to “knock in” a nucleic acid that encodes for a protein, an siRNA, an miRNA, etc.), to add a tag (e.g., 6×His, a fluorescent protein (e.g., a green fluorescent protein; a yellow fluorescent protein, etc.), hemagglutinin (HA), FLAG, etc.), to add a regulatory sequence to a gene (e.g., promoter, polyadenylation signal, internal ribosome entry sequence (IRES), 2A peptide, start codon, stop codon, splice signal, localization signal, etc.), or to modify a nucleic acid sequence (e.g., introduce a mutation).

### C. Oligonucleotide Variations

Any of the disclosed gene editing technologies, components thereof, donor oligonucleotides, or other nucleic acids can include one or more modifications or substitutions to the nucleobases or linkages. Although modifications are particularly preferred for use with triplex-forming technologies and typically discussed below with reference thereto, any of the modifications can be utilized in the construction of any of the disclosed gene editing compositions, donor oligonucleotides, other nucleotides, etc. Modifications should not prevent, and preferably enhance the activity, persistence, or function of the gene editing technology. For example, modifications to oligonucleotides for use as triplex-forming should not prevent, and preferably enhance duplex invasion, strand displacement, and/or stabilize triplex formation as described above by increasing specificity or binding affinity of the triplex-forming molecules to the target site. Modified bases and base analogues, modified sugars and sugar analogues and/or various suitable linkages known in the art are also suitable for use in the molecules disclosed herein.

i. Heterocyclic Bases

The principal naturally-occurring nucleotides include uracil, thymine, cytosine, adenine and guanine as the heterocyclic bases. Gene editing molecules can include chemical modifications to their nucleotide constituents. For example, target sequences with adjacent cytosines can be problematic. Triplex stability is greatly compromised by runs of cytosines, thought to be due to repulsion between the positive charge resulting from the N3 protonation or perhaps because of competition for protons by the adjacent cytosines. Chemical modification of nucleotides including triplex-forming molecules such as PNAs may be useful to increase binding affinity of triplex-forming molecules and/or triplex stability under physiologic conditions.

Chemical modifications of heterocyclic bases or heterocyclic base analogs may be effective to increase the binding affinity of a nucleotide or its stability in a triplex. Chemically-modified heterocyclic bases include, but are not limited to, inosine, 5-(1-propynyl) uracil (pU), 5-(1-propynyl) cytosine (pC), 5-methylcytosine, 8-oxo-adenine, pseudocytosine, pseudoisocytosine, 5 and 2-amino-5-(2′-deoxy-β-D-ribofuranosyl)pyridine (2-aminopyridine), and various pyrrolo- and pyrazolopyrimidine derivatives. Substitution of 5-methylcytosine or pseudoisocytosine for cytosine in triplex-forming molecules such as PNAs helps to stabilize triplex formation at neutral and/or physiological pH, especially in triplex-forming molecules with isolated cytosines. This is because the positive charge partially reduces the negative charge repulsion between the triplex-forming molecules and the target duplex, and allows for Hoogsteen binding.

ii. Backbone

The nucleotide subunits of the oligonucleotides may contain certain modifications. For example, the phosphate backbone of the oligonucleotide may be replaced in its entirety by repeating N-(2-aminoethyl)-glycine units and/or phosphodiester bonds may be replaced by peptide bonds or phosphorothioate linkages, either partial or complete. For example, in PNAs, the phosphate backbone of the oligonucleotide is replaced in its entirety by repeating N-(2-aminoethyl)-glycine units and phosphodiester bonds are typically replaced by peptide bonds. The various heterocyclic bases are linked to the backbone by methylene carbonyl bonds, which allow them to form PNA-DNA or PNA-RNA duplexes via Watson-Crick base pairing with high affinity and sequence-specificity. PNAs maintain spacing of heterocyclic bases that is similar to conventional DNA oligonucleotides, but are achiral and neutrally charged molecules. Peptide nucleic acids are composed of peptide nucleic acid monomers.

Other backbone modifications include peptide and amino acid variations and modifications. The backbone constituents of donor oligonucleotides may be peptide linkages, or alternatively, they may be non-peptide linkages. Examples include acetyl caps, amino spacers such as 8-amino-3,6-dioxaoctanoic acid (referred to herein as O-linkers), amino acids such as lysine are particularly useful if positive charges are desired in the oligonucleotide (e.g., PNA) and the like. Methods for the chemical assembly of PNAs are well known. See, for example, U.S. Pat. Nos. 5,539,082, 5,527,675, 5,623,049, 5,714,331, 5,736,336, 5,773,571 and 5,786,571.

Backbone modifications of oligonucleotides should not prevent the molecules from binding with high specificity to the DNA target site and mediating information transfer. For example, modifications of triplex-forming molecules should not prevent the molecules from binding with high specificity to the target site and creating a triplex with the target duplex nucleic acid by displacing one strand of the target duplex and forming a clamp around the other strand of the target duplex.

iii. Modified Nucleic Acids

Modified nucleic acids in addition to peptide nucleic acids are also useful as triplex-forming molecules. Oligonucleotides are composed of a chain of nucleotides which are linked to one another. Canonical nucleotides typically include a heterocyclic base (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic bases, and ribose or deoxyribose sugar linked by phosphodiester bonds. As used herein “modified nucleotide” or “chemically modified nucleotide” defines a nucleotide that has a chemical modification of one or more of the heterocyclic base, sugar moiety or phosphate moiety constituents. The charge of the modified nucleotide may be reduced compared to DNA or RNA oligonucleotides of the same nucleobase sequence. For example, the triplex-forming molecules may have low negative charge, no charge, or positive charge such that electrostatic repulsion with the nucleotide duplex at the target site is reduced compared to DNA or RNA oligonucleotides with the corresponding nucleobase sequence.

Examples of modified nucleotides with reduced charge include modified internucleotide linkages such as phosphate analogs having achiral and uncharged intersubunit linkages (e.g., Sterchak, E. P. et al., Organic Chem., 52:4202, (1987)), and uncharged morpholino-based polymers having achiral intersubunit linkages (see, e.g., U.S. Pat. No. 5,034,506). Some internucleotide linkage analogs include morpholidate, acetal, and polyamide-linked heterocycles. Locked nucleic acids (LNA) are modified RNA nucleotides (see, for example, Braasch, et al., Chem. Biol., 8 (1): 1-7 (2001)). LNAs form hybrids with DNA which are more stable than DNA/DNA hybrids, a property similar to that of peptide nucleic acid (PNA)/DNA hybrids. Therefore, LNA can be used just as PNA molecules would be. LNA binding efficiency can be increased in some embodiments by adding positive charges to it. Commercial nucleic acid synthesizers and standard phosphoramidite chemistry are used to make LNAs.

Molecules may also include nucleotides with modified heterocyclic bases, sugar moieties or sugar moiety analogs. Modified nucleotides may include modified heterocyclic bases or base analogs as described above with respect to peptide nucleic acids. Sugar moiety modifications include, but are not limited to, 2′-O-aminoethoxy, 2′-O amonioethyl (2′-OAE), 2′-O methoxy, 2′-O methyl, 2-guanidoethyl (2′-OGE), 2′-O,4′-C-methylene (LNA), 2′-O-(methoxyethyl) (2′-OME) and 2′-O—(N-(methyl) acetamido) (2′-OMA). 2′-O)-aminoethyl sugar moiety substitutions are especially preferred because they are protonated at neutral pH and thus suppress the charge repulsion between the triplex-forming molecule and the target duplex.

### V. Nanoparticle Delivery

Any of the disclosed compositions including, but not limited to potentiating agents, gene editing molecules, donor oligonucleotides, etc., can be delivered to the target cells using a nanoparticle delivery vehicle. In some embodiments, some of the compositions are packaged in nanoparticles and some are not. For example, in some embodiments, the gene editing technology and/or donor oligonucleotide is incorporated into nanoparticles while the potentiating agent is not. In some embodiments, the gene editing technology and/or donor oligonucleotide, and the potentiating agent are packaged in nanoparticles. The different compositions can be packaged in the same nanoparticles or different nanoparticles. For example, the compositions can be mixed and packaged together. In some embodiments, the different compositions are packaged separately into separate nanoparticles wherein the nanoparticles are similarly or identically composed and/or manufactured. In some embodiments, the different compositions are packaged separately into separate nanoparticles wherein the nanoparticles are differentially composed and/or manufactured.

Nanoparticles generally refers to particles in the range of between 500 nm to less than 0.5 nm, preferably having a diameter that is between 50 and 500 nm, more preferably having a diameter that is between 50 and 300 nm. Cellular internalization of polymeric particles is highly dependent upon their size, with nanoparticulate polymeric particles being internalized by cells with much higher efficiency than micoparticulate polymeric particles. For example, Desai, et al. have demonstrated that about 2.5 times more nanoparticles that are 100 nm in diameter are taken up by cultured Caco-2 cells as compared to microparticles having a diameter on 1 μM (Desai, et al., Pharm. Res., 14:1568-73 (1997)). Nanoparticles also have a greater ability to diffuse deeper into tissues in vivo.

### A. Polymer

The polymer that forms the core of the nanoparticle may be any biodegradable or non-biodegradable synthetic or natural polymer. In a preferred embodiment, the polymer is a biodegradable polymer.

Examples of preferred biodegradable polymers include synthetic polymers that degrade by hydrolysis such as poly(hydroxy acids), such as polymers and copolymers of lactic acid and glycolic acid, other degradable polyesters, polyanhydrides, poly(ortho) esters, polyesters, polyurethanes, poly(butic acid), poly(valeric acid), poly(caprolactone), poly(hydroxyalkanoates), poly(lactide-co-caprolactone), and poly(amine-co-ester) polymers, such as those described in Zhou, et al., Nature Materials, 11:82-90 (2012) and WO 2013/082529, U.S. Published Application No. 2014/0342003, and PCT/US2015/061375.

In some embodiments, non-biodegradable polymers can be used, especially hydrophobic polymers. Examples of preferred non-biodegradable polymers include ethylene vinyl acetate, poly(meth) acrylic acid, copolymers of maleic anhydride with other unsaturated polymerizable monomers, poly(butadiene maleic anhydride), polyamides, copolymers and mixtures thereof, and dextran, cellulose and derivatives thereof.

Other suitable biodegradable and non-biodegradable polymers are known in the art. These materials may be used alone, as physical mixtures (blends), or as co-polymers.

The nanoparticle formulation can be selected based on the considerations including the targeted tissue or cells. For example, in embodiments directed to treatment of treating or correcting beta-thalassemia (e.g. when the target cells are, for example, hematopoietic stem cells), a preferred nanoparticle formulation is PLGA. In a preferred embodiment, the nanoparticles are formed of polymers fabricated from polylactides (PLA) and copolymers of lactide and glycolide (PLGA). These have established commercial use in humans and have a long safety record (Jiang, et al., Adv. Drug Deliv. Rev., 57 (3): 391-410); Aguado and Lambert, Immunobiology, 184 (2-3): 113-25 (1992); Bramwell, et al., Adv. Drug Deliv. Rev., 57 (9): 1247-65 (2005)).

Other preferred nanoparticle formulations, particularly preferred for treating cystic fibrosis, are described in McNeer, et al., Nature Commun., 6:6952. doi: 10.1038/ncomms7952 (2015), and Fields, et al., Adv Healthc Mater., 4 (3): 361-6 (2015). doi: 10.1002/adhm.201400355 (2015) Epub 2014. Such nanoparticles are composed of a blend of Poly(beta-amino) esters (PBAEs) and poly(lactic-co-glycolic acid) (PLGA). Therefore, in some embodiments, the nanoparticles utilized to deliver the disclosed compositions are composed of a blend of PBAE and PLGA.

PLGA and PBAE/PLGA blended nanoparticles loaded with gene editing technology can be formulated using a double-emulsion solvent evaporation technique such as that described in McNeer, et al., Nature Commun., 6:6952. doi: 10.1038/ncomms7952 (2015) and Fields, et al., Adv Healthc Mater., 4 (3): 361-6 (2015). doi: 10.1002/adhm.201400355 (2015) Epub 2014. Poly(beta amino ester) (PBAE) can synthesized by a Michael addition reaction of 1,4-butanediol diacrylate and 4,4′-trimethylenedipiperidine as described in Akinc, et al., Bioconjug Chem., 14:979-988 (2003). In some embodiments, PBAE blended particles such as PLGA/PBAE blended particles, contain between about 1 and 99, or between about 1 and 50, or between about 5 and 25, or between about 5 and 20, or between about 10 and 20, or about 15 percent PBAE (wt %).

### B. Polycations

The nucleic acids can be complexed to polycations to increase the encapsulation efficiency of the nucleic acids into the nanoparticles. The term “polycation” refers to a compound having a positive charge, preferably at least 2 positive charges, at a selected pH, preferably physiological pH. Polycationic moieties have between about 2 to about 15 positive charges, preferably between about 2 to about 12 positive charges, and more preferably between about 2 to about 8 positive charges at selected pH values.

Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

Exemplary polycations include, but are not limited to, synthetic polycations based on acrylamide and 2-acrylamido-2-methylpropanetrimethylamine, poly(N-ethyl-4-vinylpyridine) or similar quartemized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyornithine.

In some embodiments, the particles themselves are a polycation (e.g., a blend of PLGA and poly(beta amino ester).

### C. Functional/Targeting Molecules

Targeting molecules can be associated with, linked, conjugated, or otherwise attached directly or indirectly to the gene editing molecule, or to a nanoparticle or other delivery vehicle thereof. Targeting molecules can be proteins, peptides, nucleic acid molecules, saccharides or polysaccharides that bind to a receptor or other molecule on the surface of a targeted cell. The degree of specificity and the avidity of binding can be modulated through the selection of the targeting molecule.

Examples of moieties include, for example, targeting moieties which provide for the delivery of molecules to specific cells, e.g., antibodies to hematopoietic stem cells, CD34⁺ cells, T cells or any other preferred cell type, as well as receptor and ligands expressed on the preferred cell type. Preferably, the moieties may target hematopoeitic stem cells. Examples of molecules targeting extracellular matrix (“ECM”) include glycosaminoglycan (“GAG”) and collagen. In one embodiment, the external surface of polymer particles may be modified to enhance the ability of the particles to interact with selected cells or tissue. In some embodiments, an adaptor element conjugated to a targeting molecule is inserted into the particle. In another embodiment, the outer surface of a polymer micro- or nanoparticle having a carboxy terminus may be linked to targeting molecules that have a free amine terminus.

Other useful ligands attached to polymeric micro- and nanoparticles include pathogen-associated molecular patterns (PAMPs). PAMPs target Toll-like Receptors (TLRs) on the surface of the cells or tissue, or signal the cells or tissue internally, thereby potentially increasing uptake. PAMPs conjugated to the particle surface or co-encapsulated may include: unmethylated CpG DNA (bacterial), double-stranded RNA (viral), lipopolysacharride (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic).

In another embodiment, the outer surface of the particle may be treated using a mannose amine, thereby mannosylating the outer surface of the particle. This treatment may cause the particle to bind to the target cell or tissue at a mannose receptor on the antigen presenting cell surface. Alternatively, surface conjugation with an immunoglobulin molecule containing an Fc portion (targeting Fc receptor), heat shock protein moiety (HSP receptor), phosphatidylserine (scavenger receptors), and lipopolysaccharide (LPS) are additional receptor targets on cells or tissue.

Lectins can be covalently attached to micro- and nanoparticles to render them target specific to the mucin and mucosal cell layer.

The choice of targeting molecule will depend on the method of administration of the nanoparticle composition and the cells or tissues to be targeted. The targeting molecule may generally increase the binding affinity of the particles for cell or tissues or may target the nanoparticle to a particular tissue in an organ or a particular cell type in a tissue. The covalent attachment of any of the natural components of mucin in either pure or partially purified form to the particles would decrease the surface tension of the bead-gut interface and increase the solubility of the bead in the mucin layer. The attachment of polyamino acids containing extra pendant carboxylic acid side groups, e.g., polyaspartic acid and polyglutamic acid, should also provide a useful means of increasing bioadhesiveness. Using polyamino acids in the 15,000 to 50,000 kDa molecular weight range yields chains of 120 to 425 amino acid residues attached to the surface of the particles. The polyamino chains increase bioadhesion by means of chain entanglement in mucin strands as well as by increased carboxylic charge.

The efficacy of the nanoparticles is determined in part by their route of administration into the body. For orally and topically administered nanoparticles, epithelial cells constitute the principal barrier that separates an organism's interior from the outside world. Therefore, in one embodiment, the nanoparticles disclosed further include epithelial cell targeting molecules, such as, antibodies or bioactive fragments thereof that recognize and bind to epitopes displayed on the surface of epithelial cells, or ligands which bind to an epithelial cell surface receptor. Examples of suitable receptors include, but are not limited to, IgE Fc receptors, EpCAM, selected carbohydrate specificites, dipeptidyl peptidase, and E-cadherin.

The efficiency of nanoparticle delivery systems can also be improved by the attachment of functional ligands to the NP surface. Potential ligands include, but are not limited to, small molecules, cell-penetrating peptides (CPPs), targeting peptides, antibodies or aptamers (Yu, et al., PLOS One., 6: e24077 (2011), Cu, et al., J Control Release, 156:258-264 (2011), Nie, et al., J Control Release, 138:64-70 (2009), Cruz, et al., J Control Release, 144:118-126 (2010)). In some embodiments, the functional molecule is a CPP such as mTAT (HIV-1 (with histidine modification) HHHHRKKRRQRRRRHHHHH (SEQ ID NO:42) (Yamano, et al., J Control Release, 152:278-285 (2011)); or bPrPp (Bovine prion) MVKSKIGSWILVLFVAMWS DVGLCKKRPKP (SEQ ID NO:43) (Magzoub, et al., Biochem Biophys Res Commun., 348:379-385 (2006)); or MPG (Synthetic chimera: SV40 Lg T. Ant. +HIV gb41 coat) GALFLGFLGAAGSTMGAWS QPKKKRKV (SEQ ID NO:44) (Endoh, et al., Adv Drug Deliv Rev., 61:704-709 (2009)). Attachment of these moieties serves a variety of different functions; such as inducing intracellular uptake, endosome disruption, and delivery to the nucleus.

### VI. Pharmaceutical Formulations

Compositions of potentiating agents (e.g., cell-penetrating anti-DNA antibody), gene editing technology, and donor oligonucleotide can be used therapeutically in combination with a suitable pharmaceutical carrier. Such compositions include an effective amount of the composition, and a pharmaceutically acceptable carrier or excipient.

It is understood by one of ordinary skill in the art that nucleotides administered in vivo are taken up and distributed to cells and tissues (Huang, et al., FEBS Lett., 558 (1-3): 69-73 (2004)). For example, Nyce, et al., have shown that antisense oligodeoxynucleotides (ODNs) when inhaled bind to endogenous surfactant (a lipid produced by lung cells) and are taken up by lung cells without a need for additional carrier lipids (Nyce, et al., Nature, 385:721-725 (1997)). Small nucleic acids are readily taken up into T24 bladder carcinoma tissue culture cells (Ma, et al., Antisense Nucleic Acid Drug Dev., 8:415-426 (1998)).

The disclosed compositions may be in a formulation for administration topically, locally or systemically in a suitable pharmaceutical carrier. Remington's Pharmaceutical Sciences, 15th Edition by E. W. Martin (Mark Publishing Company, 1975), discloses typical carriers and methods of preparation. The compound may also be encapsulated in suitable biocompatible microcapsules, microparticles, nanoparticles, or microspheres formed of biodegradable or non-biodegradable polymers or proteins or liposomes for targeting to cells. Such systems are well known to those skilled in the art and may be optimized for use with the appropriate nucleic acid. As described above, in some embodiments, the donor oligonucleotide is encapsulated in nanoparticles.

Various methods for nucleic acid delivery are described, for example, in Sambrook et al., Molecular Cloning: A Laboratory Manual, Cold Spring Harbor Laboratory, New York (1989); and Ausubel, et al., Current Protocols in Molecular Biology, John Wiley & Sons, New York (1994). Such nucleic acid delivery systems include the desired nucleic acid, by way of example and not by limitation, in either “naked” form as a “naked” nucleic acid, or formulated in a vehicle suitable for delivery, such as in a complex with a cationic molecule or a liposome forming lipid, or as a component of a vector, or a component of a pharmaceutical composition. The nucleic acid delivery system can be provided to the cell either directly, such as by contacting it with the cell, or indirectly, such as through the action of any biological process. The nucleic acid delivery system can be provided to the cell by endocytosis, receptor targeting, coupling with native or synthetic cell membrane fragments, physical means such as electroporation, combining the nucleic acid delivery system with a polymeric carrier such as a controlled release film or nanoparticle or microparticle, using a vector, injecting the nucleic acid delivery system into a tissue or fluid surrounding the cell, simple diffusion of the nucleic acid delivery system across the cell membrane, or by any active or passive transport mechanism across the cell membrane. Additionally, the nucleic acid delivery system can be provided to the cell using techniques such as antibody-related targeting and antibody-mediated immobilization of a viral vector.

Formulations for injection may be presented in unit dosage form, e.g., in ampules or in multi-dose containers, optionally with an added preservative. The compositions may take such forms as sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). The materials may be in solution, emulsions, or suspension (for example, incorporated into particles, liposomes, or cells). Typically, an appropriate amount of a pharmaceutically-acceptable salt is used in the formulation to render the formulation isotonic. Trehalose, typically in the amount of 1-5%, may be added to the pharmaceutical compositions. The pH of the solution is preferably from about 5 to about 8, and more preferably from about 7 to about 7.5. Pharmaceutical compositions may include carriers, thickeners, diluents, buffers, preservatives, and surface-active agents. Carrier formulation can be found in Remington's Pharmaceutical Sciences, Mack Publishing Co., Easton, Pa. Those of skill in the art can readily determine the various parameters for preparing and formulating the compositions without resort to undue experimentation.

The disclosed compositions alone or in combination with other suitable components, can also be made into aerosol formulations (i.e., they can be “nebulized”) to be administered via inhalation. Aerosol formulations can be placed into pressurized acceptable propellants, such as dichlorodifluoromethane, propane, nitrogen, and air. For administration by inhalation, the compounds are delivered in the form of an aerosol spray presentation from pressurized packs or a nebulizer, with the use of a suitable propellant.

In some embodiments, the compositions include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers. Trehalose, typically in the amount of 1-5%, may be added to the pharmaceutical compositions. The donor oligonucleotides may be conjugated to lipophilic groups like cholesterol and lauric and lithocholic acid derivatives with C32 functionality to improve cellular uptake. For example, cholesterol has been demonstrated to enhance uptake and serum stability of siRNA in vitro (Lorenz, et al., Bioorg. Med. Chem. Lett., 14 (19): 4975-4977 (2004)) and in vivo (Soutschek, et al., Nature, 432 (7014): 173-178 (2004)). In addition, it has been shown that binding of steroid conjugated oligonucleotides to different lipoproteins in the bloodstream, such as LDL, protect integrity and facilitate biodistribution (Rump, et al., Biochem. Pharmacol., 59 (11): 1407-1416 (2000)). Other groups that can be attached or conjugated to the compound described above to increase cellular uptake, include acridine derivatives; cross-linkers such as psoralen derivatives, azidophenacyl, proflavin, and azidoproflavin; artificial endonucleases; metal complexes such as EDTA-Fe (II) and porphyrin-Fe(II); alkylating moieties; nucleases such as alkaline phosphatase; terminal transferases; abzymes; cholesteryl moieties; lipophilic carriers; peptide conjugates; long chain alcohols; phosphate esters; radioactive markers; non-radioactive markers; carbohydrates; and polylysine or other polyamines. U.S. Pat. No. 6,919,208 to Levy, et al., also describes methods for enhanced delivery. These pharmaceutical formulations may be manufactured in a manner that is itself known, e.g., by means of conventional mixing, dissolving, granulating, levigating, emulsifying, encapsulating, entrapping or lyophilizing processes.

Further carriers include sustained release preparations such as semi-permeable matrices of solid hydrophobic polymers containing the antibody, which matrices are in the form of shaped particles, e.g., films, liposomes or microparticles. Implantation includes inserting implantable drug delivery systems, e.g., microspheres, hydrogels, polymeric reservoirs, cholesterol matrixes, polymeric systems, e.g., matrix erosion and/or diffusion systems and non-polymeric systems, e.g., compressed, fused, or partially-fused pellets. Inhalation includes administering the composition with an aerosol in an inhaler, either alone or attached to a carrier that can be absorbed. For systemic administration, it may be preferred that the composition is encapsulated in liposomes.

The compositions may be delivered in a manner which enables tissue-specific uptake of the agent and/or nucleotide delivery system. Techniques include using tissue or organ localizing devices, such as wound dressings or transdermal delivery systems, using invasive devices such as vascular or urinary catheters, and using interventional devices such as stents having drug delivery capability and configured as expansive devices or stent grafts.

Formulations of the compositions (e.g., containing the cell-penetrating antibody, gene editing technology and donor oligonucleotide) may be delivered using a bioerodible implant by way of diffusion or by degradation of the polymeric matrix. In certain embodiments, the administration of the formulation may be designed so as to result in sequential exposures to the composition, over a certain time period, for example, hours, days, weeks, months or years. This may be accomplished, for example, by repeated administrations of a formulation or by a sustained or controlled release delivery system in which the compositions are delivered over a prolonged period without repeated administrations.

Suitable delivery systems include time-release, delayed release, sustained release, or controlled release delivery systems. Such systems may avoid repeated administrations in many cases, increasing convenience to the subject and the physician. Many types of release delivery systems are available and known to those of ordinary skill in the art. They include, for example, polymer-based systems such as polylactic and/or polyglycolic acids, polyanhydrides, polycaprolactones, copolyoxalates, polyesteramides, polyorthoesters, polyhydroxybutyric acid, and/or combinations of these. Microcapsules of the foregoing polymers containing nucleic acids are described in, for example, U.S. Pat. No. 5,075,109. Other examples include non-polymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulations containing the potentiating agent, gene editing technology and/or donor oligonucleotide.

Active agent(s) (potentiating agent, gene editing technology and donor oligonucleotide) and compositions thereof can be formulated for pulmonary or mucosal administration. The administration can include delivery of the composition to the lungs, nasal, oral (sublingual, buccal), vaginal, or rectal mucosa. The term aerosol as used herein refers to any preparation of a fine mist of particles, which can be in solution or a suspension, whether or not it is produced using a propellant. Aerosols can be produced using standard techniques, such as ultrasonication or high-pressure treatment.

For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers.

### VII. Methods

The disclosed compositions can be used for in vitro, ex vivo or in vivo gene editing. The methods typically include contacting a cell with an effective amount of gene editing composition, in combination with a potentiating agent, to modify the cell's genome. In preferred embodiments, the method includes contacting a population of target cells with an effective amount of gene editing composition and donor oligonucleotide, in combination with a potentiating agent (e.g., cell-penetrating antibody), to modify the genomes of a sufficient number of cells to achieve a therapeutic result.

Potentiating agent and gene editing composition can be contacted with the cells together in the same or different admixtures, or potentiating agent and gene editing composition can be contacted with cells separately. For example, cells can be first contacted with potentiating agent, followed by gene editing composition. Alternatively, cells can be first contacted with gene editing composition, followed by potentiating agent. In some embodiments, gene editing composition and potentiating agent are mixed in solution and contacted with cells simultaneously. In a preferred embodiment, gene editing composition is mixed with potentiating agent in solution and the combination is added to the cells in culture or injected into an animal to be treated.

The effective amount or therapeutically effective amount can be a dosage sufficient to treat, inhibit, or alleviate one or more symptoms of a disease or disorder, or to otherwise provide a desired pharmacologic and/or physiologic effect, for example, reducing, inhibiting, or reversing one or more of the pathophysiological mechanisms underlying a disease or disorder.

In some embodiments, when the gene editing technology is triplex-forming molecules, the molecules can be administered in an effective amount to induce formation of a triple helix at the target site. An effective amount of gene editing technology such as triplex-forming molecules may also be an amount effective to increase the rate of recombination of a donor fragment relative to administration of the donor fragment in the absence of the gene editing technology. The formulation of the potentiating agent, gene editing technology, and donor oligonucleotide is made to suit the mode of administration. Pharmaceutically acceptable carriers are determined in part by the particular composition being administered, as well as by the particular method used to administer the composition. Accordingly, there is a wide variety of suitable formulations of pharmaceutical compositions containing the potentiating agent, gene editing technology, and donor oligonucleotide. The precise dosage will vary according to a variety of factors such as subject-dependent variables (e.g., age, immune system health, clinical symptoms etc.).

The disclosed compositions can be administered or otherwise contacted with target cells once, twice, or three time daily; one, two, three, four, five, six, seven times a week, one, two, three, four, five, six, seven or eight times a month. For example, in some embodiments, the composition is administered every two or three days, or on average about 2 to about 4 times about week.

The compositions may or may not be administered at the same time. In some embodiments, the potentiating agent (e.g., cell-penetrating antibody) is administered to the subject prior to administration of the gene editing technology and/or donor oligonucleotide to the subject. The potentiating agent can be administered to the subject, for example, 1, 2, 3, 4, 5, 6, 8, 10, 12, 18, or 24 hours, or 1, 2, 3, 4, 5, 6, or 7 days, or any combination thereof prior to administration of the gene editing technology and/or donor oligonucleotide to the subject.

In some embodiments, the gene editing technology and/or donor oligonucleotide is administered to the subject prior to administration of the potentiating agent to the subject. The gene editing technology can be administered to the subject, for example, 1, 2, 3, 4, 5, 6, 8, 10, 12, 18, or 24 hours, or 1, 2, 3, 4, 5, 6, or 7 days, or any combination thereof prior to administration of the potentiating agent to the subject.

In some embodiments, the potentiating agent (e.g., cell-penetrating antibody) and donor oligonucleotide can be contacted with the cells together in the same or different admixtures, separate from the gene editing technology (e.g., PNA or CRISPR/Cas). In some embodiments, the potentiating agent (e.g., cell-penetrating antibody) and donor oligonucleotide can be contacted with cells separately. For example, in some embodiments, donor oligonucleotide and the potentiating agent (e.g., cell-penetrating antibody) may be mixed in solution and contacted with cells simultaneously, which may be separate from contacting of the cells with the gene editing technology (e.g., PNA or CRISPR/Cas).

In preferred embodiments, the potentiating agent and donor oligonucleotide are administered in an amount effective to induce gene modification in at least one target allele to occur at frequency of at least 0.01, 0.02. 0.03, 0.04, 0.05, 0.06, 0.07, 0.08, 0.09, 0.1, 0.2. 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, or 25% of target cells. In some embodiments, particularly ex vivo applications, gene modification occurs in at least one target allele at a frequency of about 0.1-25%, or 0.5-25%, or 1-25% 2-25%, or 3-25%, or 4-25% or 5-25% or 6-25%, or 7-25%, or 8-25%, or 9-25%, or 10-25%, 11-25%, or 12-25%, or 13%-25% or 14%-25% or 15-25%, or 2-20%, or 3-20%, or 4-20% or 5-20% or 6-20%, or 7-20%, or 8-20%, or 9-20%, or 10-20%, 11-20%, or 12-20%, or 13%-20% or 14%-20% or 15-20%, 2-15%, or 3-15%, or 4-15% or 5-15% or 6-15%, or 7-15%, or 8-15%, or 9-15%, or 10-15%, 11-15%, or 12-15%, or 13%-15% or 14%-15%.

In some embodiments, particularly in vivo applications, gene modification occurs in at least one target allele at a frequency of about 0.1% to about 15%, or about 0.2% to about 15%, or about 0.3% to about 15%, or about 0.4% to about 15%, or about 0.5% to about 15%, or about 0.6% to about 15%, or about 0.7% to about 15%, or about 0.8% to about 15%, or about 0.9% to about 15%, or about 1.0% to about 15%, or about 1.1% to about 15%, or about 1.1% to about 15%, 1.2% to about 15%, or about 1.3% to about 15%, or about 1.4% to about 15%, or about 1.5% to about 15%, or about 1.6% to about 15%, or about 1.7% to about 15%, or about 1.8% to about 15%, or about 1.9% to about 15%, or about 2.0% to about 15%, or about 2.5% to about 15%, or about 3.0% to about 15%, or about 3.5% to about 15%, or about 4.0% to about 15%, or about 4.5% to about 15%, or about 5.0% to about 15%, or about 1% to about 15%, about 1.5% to about 15%, about 2.0% to about 15%, or about 2.5% to about 15%, or about 3.0% to about 15%, or about 3.5% to about 15%, or about 4.0% to about 15%, or about 4.5% to about 15%.

In some embodiments, gene modification occurs with low off-target effects. In some embodiments, off-target modification is undetectable using routine analysis such as, but not limited to, those described in the Examples. In some embodiments, off-target incidents occur at a frequency of 0-1%, or 0-0.1%, or 0-0.01%, or 0-0.001%, or 0-0.0001%, or 0-0000.1%, or 0-0.000001%. In some embodiments, off-target modification occurs at a frequency that is about 10², 10³, 10⁴, or 10⁵-fold lower than at the target site.

### A. Ex Vivo Gene Therapy

In some embodiments, ex vivo gene therapy of cells is used for the treatment of a genetic disorder in a subject. For ex vivo gene therapy, cells are isolated from a subject and contacted ex vivo with the compositions (potentiating agent, gene editing technology, and/or donor oligonucleotide) to produce cells containing altered sequences in or adjacent to genes. In a preferred embodiment, the cells are isolated from the subject to be treated or from a syngenic host. Target cells are removed from a subject prior to contacting with a gene editing composition and a potentiating agent. The cells can be hematopoietic progenitor or stem cells. In a preferred embodiment, the target cells are CD34⁺ hematopoietic stem cells. Hematopoietic stem cells (HSCs), such as CD34+ cells are multipotent stem cells that give rise to all the blood cell types including erythrocytes. Therefore, CD34+ cells can be isolated from a patient with, for example, thalassemia, sickle cell disease, or a lysosomal storage disease, the mutant gene altered or repaired ex-vivo using the disclosed compositions and methods, and the cells reintroduced back into the patient as a treatment or a cure.

Stem cells can be isolated and enriched by one of skill in the art. Methods for such isolation and enrichment of CD34+ and other cells are known in the art and disclosed for example in U.S. Pat. Nos. 4,965,204; 4,714,680; 5,061,620; 5,643,741; 5,677,136; 5,716,827; 5,750,397 and 5,759,793. As used herein in the context of compositions enriched in hematopoietic progenitor and stem cells, “enriched” indicates a proportion of a desirable element (e.g. hematopoietic progenitor and stem cells) which is higher than that found in the natural source of the cells. A composition of cells may be enriched over a natural source of the cells by at least one order of magnitude, preferably two or three orders, and more preferably 10, 100, 200 or 1000 orders of magnitude.

In humans, CD34⁺ cells can be recovered from cord blood, bone marrow or from blood after cytokine mobilization effected by injecting the donor with hematopoietic growth factors such as granulocyte colony stimulating factor (G-CSF), granulocyte-monocyte colony stimulating factor (GM-CSF), stem cell factor (SCF) subcutaneously or intravenously in amounts sufficient to cause movement of hematopoietic stem cells from the bone marrow space into the peripheral circulation. Initially, bone marrow cells may be obtained from any suitable source of bone marrow, e.g. tibiae, femora, spine, and other bone cavities. For isolation of bone marrow, an appropriate solution may be used to flush the bone, which solution will be a balanced salt solution, conveniently supplemented with fetal calf serum or other naturally occurring factors, in conjunction with an acceptable buffer at low concentration, generally from about 5 to 25 mM. Convenient buffers include Hepes, phosphate buffers, lactate buffers, etc.

Cells can be selected by positive and negative selection techniques. Cells can be selected using commercially available antibodies which bind to hematopoietic progenitor or stem cell surface antigens, e.g. CD34, using methods known to those of skill in the art. For example, the antibodies may be conjugated to magnetic beads and immunogenic procedures utilized to recover the desired cell type. Other techniques involve the use of fluorescence activated cell sorting (FACS). The CD34 antigen, which is found on progenitor cells within the hematopoietic system of non-leukemic individuals, is expressed on a population of cells recognized by the monoclonal antibody My-10 (i.e., express the CD34 antigen) and can be used to isolate stem cell for bone marrow transplantation. My-10 deposited with the American Type Culture Collection (Rockville, Md.) as HB-8483 is commercially available as anti-HPCA 1. Additionally, negative selection of differentiated and “dedicated” cells from human bone marrow can be utilized, to select against substantially any desired cell marker. For example, progenitor or stem cells, most preferably CD34′ cells, can be characterized as being any of CD3⁻, CD7⁻, CD8⁻, CD10⁻, CD14⁻, CD15⁻, CD19⁻, CD20⁻, CD33⁻, Class II HLA⁺ and Thy-1⁺.

Once progenitor or stem cells have been isolated, they may be propagated by growing in any suitable medium. For example, progenitor or stem cells can be grown in conditioned medium from stromal cells, such as those that can be obtained from bone marrow or liver associated with the secretion of factors, or in medium including cell surface factors supporting the proliferation of stem cells. Stromal cells may be freed of hematopoietic cells employing appropriate monoclonal antibodies for removal of the undesired cells.

The isolated cells are contacted ex vivo with a combination of a gene editing technology, potentiating agent and donor oligonucleotides in amounts effective to cause the desired alterations in or adjacent to genes in need of repair or alteration, for example the human beta-globin or «-L-iduronidase gene. These cells are referred to herein as modified cells. Methods for transfection of cells with oligonucleotides are well known in the art (Koppelhus, et al., Adv. Drug Deliv. Rev., 55 (2): 267-280 (2003)). It may be desirable to synchronize the cells in S-phase to further increase the frequency of gene correction. Methods for synchronizing cultured cells, for example, by double thymidine block, are known in the art (Zielke, et al., Methods Cell Biol., 8:107-121 (1974)).

The modified cells can be maintained or expanded in culture prior to administration to a subject. Culture conditions are generally known in the art depending on the cell type. Conditions for the maintenance of CD34⁺ in particular have been well studied, and several suitable methods are available. A common approach to ex vivo multi-potential hematopoietic cell expansion is to culture purified progenitor or stem cells in the presence of early-acting cytokines such as interleukin-3. It has also been shown that inclusion, in a nutritive medium for maintaining hematopoietic progenitor cells ex vivo, of a combination of thrombopoietin (TPO), stem cell factor (SCF), and flt3 ligand (Flt-3L; i.e., the ligand of the flt3 gene product) was useful for expanding primitive (i.e., relatively non-differentiated) human hematopoietic progenitor cells in vitro, and that those cells were capable of engraftment in SCID-hu mice (Luens et al., 1998, Blood 91:1206-1215). In other known methods, cells can be maintained ex vivo in a nutritive medium (e.g., for minutes, hours, or 3, 6, 9, 13, or more days) including murine prolactin-like protein E (mPLP-E) or murine prolactin-like protein F (mPIP-F; collectively mPLP-E/IF) (U.S. Pat. No. 6,261,841). It will be appreciated that other suitable cell culture and expansion methods can be used as well. Cells can also be grown in serum-free medium, as described in U.S. Pat. No. 5,945,337.

In another embodiment, the modified hematopoietic stem cells are differentiated ex vivo into CD4⁺ cells culture using specific combinations of interleukins and growth factors prior to administration to a subject using methods well known in the art. The cells may be expanded ex vivo in large numbers, preferably at least a 5-fold, more preferably at least a 10-fold and even more preferably at least a 20-fold expansion of cells compared to the original population of isolated hematopoietic stem cells.

In another embodiment cells, for ex vivo gene therapy can be dedifferentiated somatic cells. Somatic cells can be reprogrammed to become pluripotent stem-like cells that can be induced to become hematopoietic progenitor cells. The hematopoietic progenitor cells can then be treated with a potentiating agent, gene editing technology and donor oligonucleotide to produce recombinant cells having one or more modified genes. Representative somatic cells that can be reprogrammed include, but are not limited to fibroblasts, adipocytes, and muscles cells. Hematopoietic progenitor cells from induced stem-like cells have been successfully developed in the mouse (Hanna, J. et al. Science, 318:1920-1923 (2007)).

To produce hematopoietic progenitor cells from induced stem-like cells, somatic cells are harvested from a host. In a preferred embodiment, the somatic cells are autologous fibroblasts. The cells are cultured and transduced with vectors encoding Oct4, Sox2, Klf4, and c-Myc transcription factors. The transduced cells are cultured and screened for embryonic stem cell (ES) morphology and ES cell markers including, but not limited to AP, SSEA1, and Nanog. The transduced ES cells are cultured and induced to produce induced stem-like cells. Cells are then screened for CD41 and c-kit markers (early hematopoietic progenitor markers) as well as markers for myeloid and erythroid differentiation.

The modified hematopoietic stem cells or modified induced hematopoietic progenitor cells are then introduced into a subject. Delivery of the cells may be affected using various methods and includes most preferably intravenous administration by infusion as well as direct depot injection into periosteal, bone marrow and/or subcutaneous sites.

The subject receiving the modified cells may be treated for bone marrow conditioning to enhance engraftment of the cells. The recipient may be treated to enhance engraftment, using a radiation or chemotherapeutic treatment prior to the administration of the cells. Upon administration, the cells will generally require a period of time to engraft. Achieving significant engraftment of hematopoietic stem or progenitor cells typically takes weeks to months.

A high percentage of engraftment of modified hematopoietic stem cells is not envisioned to be necessary to achieve significant prophylactic or therapeutic effect. It is believed that the engrafted cells will expand over time following engraftment to increase the percentage of modified cells. For example, in some embodiments, the modified cells have a corrected cx-L-iduronidase gene. Therefore, in a subject with Hurler syndrome, the modified cells can improve or cure the condition. It is believed that engraftment of only a small number or small percentage of modified hematopoietic stem cells will be required to provide a prophylactic or therapeutic effect.

In preferred embodiments, the cells to be administered to a subject will be autologous, e.g. derived from the subject, or syngenic.

In some embodiments, the compositions and methods can be used to edit embryonic genomes in vitro. The methods typically include contacting an embryo in vitro with an effective amount of potentiating agent and gene editing technology to induce at least one alteration in the genome of the embryo. Most preferably the embryo is a single cell zygote, however, treatment of male and female gametes prior to and during fertilization, and embryos having 2, 4, 8, or 16 cells and including not only zygotes, but also morulas and blastocytes, are also provided. Typically, the embryo is contacted with the compositions on culture days 0-6 during or following in vitro fertilization.

The contacting can be adding the compositions to liquid media bathing the embryo. For example, the compositions can be pipetted directly into the embryo culture media, whereupon they are taken up by the embryo.

### B. In Vivo Gene Therapy

In some embodiments, in vivo gene therapy of cells is used for the treatment of a genetic disorder in a subject. The disclosed compositions can be administered directly to a subject for in vivo gene therapy.

In general, methods of administering compounds, including antibodies, oligonucleotides and related molecules, are well known in the art. In particular, the routes of administration already in use for nucleic acid therapeutics, along with formulations in current use, provide preferred routes of administration and formulation for the donor oligonucleotides described above. Preferably the compositions are injected or infused into the organism undergoing genetic manipulation, such as an animal requiring gene therapy.

The disclosed compositions can be administered by a number of routes including, but not limited to, intravenous, intraperitoneal, intraamniotic, intramuscular, subcutaneous, or topical (sublingual, rectal, intranasal, pulmonary, rectal mucosa, and vaginal), and oral (sublingual, buccal).

In some embodiments, the compounds are formulated for pulmonary delivery, such as intranasal administration or oral inhalation. Administration of the formulations may be accomplished by any acceptable method that allows the potentiating agent, gene editing technology, and/or donor oligonucleotide to reach their targets. The administration may be localized (i.e., to a particular region, physiological system, tissue, organ, or cell type) or systemic, depending on the condition being treated. Compositions and methods for in vivo delivery are also discussed in WO 2017/143042.

The methods can also include administering an effective amount of potentiating agent and gene editing technology to an embryo or fetus, or the pregnant mother thereof, in vivo. In some methods, compositions are delivered in utero by injecting and/or infusing the compositions into a vein or artery, such as the vitelline vein or the umbilical vein, or into the amniotic sac of an embryo or fetus. See, e.g., Ricciardi, et al., Nat Commun. 2018 Jun. 26; 9 (1): 2481. doi: 10.1038/s41467-018-04894-2, and WO 2018/187493.

### C. Diseases to be Treated

Gene therapy is apparent when studied in the context of human genetic diseases, for example, cystic fibrosis, hemophilia, musclular dystrophy, globinopathies such as sickle cell anemia and beta-thalassemia, xeroderma pigmentosum, and lysosomal storage diseases, though the strategies are also useful for treating non-genetic disease such as HIV, in the context of ex vivo-based cell modification and also for in vivo cell modification. The methods using potentiating agents, gene editing technology, and/or donor oligonucleotides are especially useful to treat genetic deficiencies, disorders and diseases caused by mutations in single genes, for example, to correct genetic deficiencies, disorders and diseases caused by point mutations. If the target gene contains a mutation that is the cause of a genetic disorder, then the disclosed methods can be used for mutagenic repair that may restore the DNA sequence of the target gene to normal. The target sequence can be within the coding DNA sequence of the gene or within an intron. The target sequence can also be within DNA sequences that regulate expression of the target gene, including promoter or enhancer sequences.

In the methods disclosed, cells that have been contacted with the potentiating agent, gene editing technology and/or donor oligonucleotide may be administered to a subject. The subject may have a disease or disorder such as hemophilia, muscular dystrophy, globinopathies, cystic fibrosis, xeroderma pigmentosum, lysosomal storage diseases, immune deficiency syndromes such as X-linked severe combined immunodeficiency and ADA deficiency, tyrosinemia, Fanconi anemia, the red cell disorder spherocytosis, alpha-1-anti-trypsin deficiency, Wilson's disease, Leber's hereditary optic neuropathy, or chronic granulomatous disorder. In such embodiments, gene modification may occur in an effective amount to reduce one or more symptoms of the disease or disorder in the subject. Exemplary sequences for triplex-forming molecules and donor oligonucleotides designed to correct mutations in globinopathies, cystic fibrosis, HIV, and lysosomal storage diseases are known in the art and disclosed in, for example, published international applications WO 2017/143042, WO 2017/143061, WO 2018/187493, and published U.S. Application No. 2017/0283830, each of which is specifically incorporated by reference in its entirety.

### D. Combination Therapies

Each of the different components for gene editing disclosed here can be administered alone or in any combination and further in combination with one or more additional active agents. In all cases, the combination of agents can be part of the same admixture, or administered as separate compositions. In some embodiments, the separate compositions are administered through the same route of administration. In other embodiments, the separate compositions are administered through different routes of administration.

Examples of preferred additional active agents include other conventional therapies known in the art for treating the desired disease or condition. For example, in the treatment of sickle cell disease, the additional therapy may be hydroxyurea.

In the treatment of cystic fibrosis, the additional therapy may include mucolytics, antibiotics, nutritional agents, etc. Specific drugs are outlined in the Cystic Fibrosis Foundation drug pipeline and include, but are not limited to, CFTR modulators such as KALYDECO® (ivacaftor), ORKAMBI™ (lumacaftor+ivacaftor), ataluren (PTC124), VX-661+invacaftor, riociguat, QBW251, N91115, and QR-010; agents that improve airway surface liquid such as hypertonic saline, bronchitol, and P-1037; mucus alteration agents such as PULMOZYME® (dornase alfa); anti-inflammatoires such as ibuprofen, alpha 1 anti-trypsin, CTX-4430, and JBT-101; anti-infective such as inhaled tobramycin, azithromycin, CAYSTON® (aztreonam for inhalation solution), TOBI inhaled powder, levofloxacin, ARIKACE® (nebulized liposomal amikacin), AEROVANC® (vancomycin hydrochloride inhalation powder), and gallium; and nutritional supplements such as aquADEKs, pancrelipase enzyme products, liprotamase, and burlulipase.

In the treatment of HIV, the additional therapy maybe an antiretroviral agents including, but not limited to, a non-nucleoside reverse transcriptase inhibitor (NNRTIs), a nucleoside reverse transcriptase inhibitor (NRTIs), a protease inhibitors (PIs), a fusion inhibitors, a CCR5 antagonists (CCR5s) (also called entry inhibitors), an integrase strand transfer inhibitors (INSTIs), or a combination thereof.

In the treatment of lysosomal storage disease, the additional therapy could include, for example, enzyme replacement therapy, bone marrow transplantation, or a combination thereof.

### E. Determining Gene Modification

Sequencing and allele-specific PCR are preferred methods for determining if gene modification has occurred. PCR primers are designed to distinguish between the original allele, and the new predicted sequence following recombination. Other methods of determining if a recombination event has occurred are known in the art and may be selected based on the type of modification made. Methods include, but are not limited to, analysis of genomic DNA, for example by sequencing, allele-specific PCR, droplet digital PCR, or restriction endonuclease selective PCR (REMS-PCR); analysis of mRNA transcribed from the target gene for example by Northern blot, in situ hybridization, real-time or quantitative reverse transcriptase (RT) PCR; and analysis of the polypeptide encoded by the target gene, for example, by immunostaining, ELISA, or FACS. In some cases, modified cells will be compared to parental controls. Other methods may include testing for changes in the function of the RNA transcribed by, or the polypeptide encoded by the target gene. For example, if the target gene encodes an enzyme, an assay designed to test enzyme function may be used.

### EXAMPLES

### Example 1: Rad51 Knockdown Enhances PNA-Mediated Gene Editing in K562 Cells

### Materials and Methods

### PNA and Donor DNA

The sequence of the triplex forming PNA (designated PNA194) was H-KKK-JJTJTTJTT-O—O—O-TTCTTCTCC—KKK-NH₂, (SEQ ID NO:45) where, J=pseudoisocytosine, K=lysine, and O=flexible octanoic acid linker.

The single-stranded donor DNA oligomer was prepared by standard DNA synthesis and 5′ and 3′-end protected by inclusion of three phosphorothioate internucleoside linkages at each end. The sequence of the donor DNA was

(SEQ ID NO: 46)
5′GTTCAGCGTGTCCGGCGAGGGCGAGGTGAGTCTATGGGACCCT
TGATGTTT 3′ (51 nucleotides).

### Cell Culture and Treatment

A cell culture model of human K562 cells was used. These cells carry a β-globin/GFP fusion transgene consisting of human β-globin intron 2 carrying a thalassemia-associated IVS2-1 (G→A) mutation embedded within the GFP coding sequence, resulting in incorrect splicing of β-globin/GFP mRNA and lack of GFP expression (Chin, et al., Proc Natl Acad Sci USA, 105 (36): 13514-9 (2008)). Correction of the mutation can be scored by green fluorescence, by DNA sequencing, allele specific PCR, or droplet digital PCR.

K562 cells were treated with SMARTpool siRNAs (Dharmacon) to achieve knockdown of specific DNA repair factors. The cells were grown in RPMI medium supplemented with 10% fetal bovine serum. 48 hours later, the cells were nucleofected with PNAs and single-stranded donor DNAs.

48 hours later, genomic DNA was isolated and allele-specific PCR was used to measure successful gene editing to correct the IVS2-1 mutation.

### Results

The impact of siRNA knockdown of DNA repair factors on PNA-mediated gene editing in human K562 cells was investigated. Western blot analysis demonstrated complete knockdown of RAD51 protein at 72 hours post-transfection. Gene-editing in the knockdown cell populations was then analyzed by allele-specific PCR to quantify gene editing in a GFP-β-globin fusion gene model.

The PCR results demonstrated that RAD51 was not required for PNA-mediated gene editing. It was also observed that siRNA knockdown of RAD51 actually boosted the efficiency of editing, as measured by allele-specific PCR. In contrast, knockdown of the related recombinase, RAD52, suppressed PNA-mediated gene editing. Similar experiments demonstrated that knockdown of XPA, FANCD2, FANCA, and XRCCI all led to suppression of PNA-mediated gene editing. Like knockdown of RAD51, knockdown of XRCC4 enhanced gene editing.

### Example 2: 3E10 Enhances Editing of the Beta Globin Gene Both Ex Vivo and In Vivo Using the β-Globin/GFP Mouse Model

### Materials and Methods

### PNA and Donor DNA

The single-stranded donor DNA oligomer was prepared by standard DNA synthesis and 5′ and 3′-end protected by inclusion of three phosphorothioate internucleoside linkages at each end. The sequence of the donor DNA matches positions 624 to 684 in β-globin intron 2 and is as follows, with the correcting IVS2-654 nucleotide underlined:

(SEQ ID NO: 47)
5′AAAGAATAACAGTGATAATTTCTGGGTTAAGGCAATAGCAATATCTCT
GCATATAAATAT3′.

The sequence of the PNA (designated ytcPNA4) was H-KKK-JTTTJTTTJTJT-OOO-TCTCTTTCTTTCAGGGCA-KKK-NH₂ (SEQ ID NO: 48), where the underlined nucleobases have a gamma mini-PEG side chain substitution, J-pseudoisocytosine, K=lysine, and O=flexible octanoic acid linker.

### Nanoparticle Synthesis

The polymeric PLGA nanoparticles used to deliver the gene editing agents were synthesized by a double-emulsion solvent evaporation protocol as previously described (Bahal, et al., Nat. Commun., 7:13304 (2016)).

### Mouse Model

Gene editing was evaluated in murine embryonic fibroblasts (MEFs) from mice carrying a β-globin/GFP fusion transgene consisting of human β-globin intron 2 carrying a different thalassemia-associated IVS2-654 (C→T) mutation embedded within the GFP coding sequence, resulting in incorrect splicing of β-globin/GFP mRNA and lack of GFP expression (Chin, et al., Proc Natl Acad Sci USA, 105 (36): 13514-9 (2008)). Correction of the IVS2-654 (C→T) mutation by gene editing causes the cells to express a functional GFP and appear green, which is quantified by flow cytometry

### Cell Culture and Treatment

To evaluate the effects of 3E10 on PNA/DNA directed gene editing ex vivo, MEFs (isolated from the β-globin/GFP transgenic mouse model described above) were treated with nanoparticles containing PNA plus donor DNA by simple addition to the cell culture (DMEM media, containing 10% FBS). Cells were seeded at 2500 cells/well. The cells were treated when sub-confluent. The cells were then analyzed for gene editing 72 h later by fluorescence via flow cytometry.

In some samples, 24 h prior to treatment with 2 mg of donor DNA nanoparticles, cells were treated either with siRNA to RAD51, a scrambled, control siRNA, or with 3E10 (at the indicated doses).

Gene-edited MEF populations were then analyzed by FACS to identify the frequency of editing using the GFP read out in the GFP-β-globin fusion gene model.

### Mouse Treatment

To evaluate the effects of 3E10 on PNA/DNA directed gene editing in vivo, the same β-globin/GFP transgenic mouse model described above was used. Three hours prior to treatment with nanoparticles, mice were injected with 0.5 mg of 3E10 intraperitoneally (i.p.). Either the full-length 3E10 or a single-chain variable fragment (scFv) were used. Two mg of nanoparticles containing PNA/Donor DNA were then injected intravenously. After eight days, bone marrow and spleens were harvested and CD117+ cells (C-KIT+, a marker of hematopoietic stem and progenitor cells) from these tissues were isolated using a Hematopoietic Progenitor Stem Cell Enrichment Set (BD Bioscience). Following enrichment, cells were analyzed via flow cytometry for GFP expression.

### Results

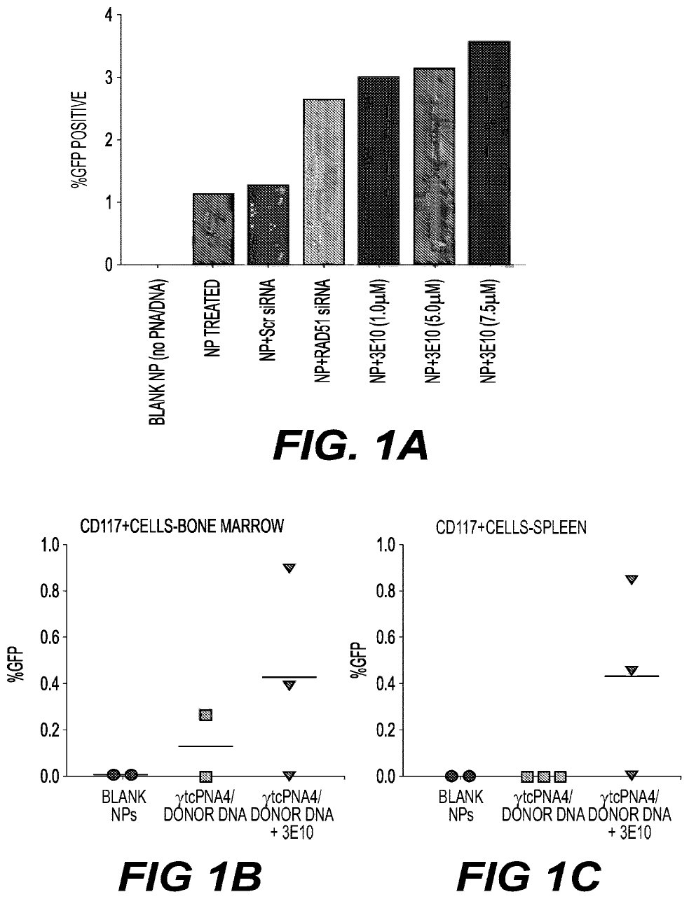

As shown in FIG. 1A, RAD51 siRNA pre-treatment prior to nanoparticle delivery of PNA/DNA resulted in a 2.4-fold increase in editing efficiency, as compared to cells with no siRNA treatment. Such an effect was not observed in the pre-treatment by scramble-sequence siRNA control. Pre-treatment with 3E10 at 24 hours prior to nanoparticle treatment of the cells resulted in a dose-dependent effect, with a range of 2.7 to 3.2-fold gene editing increases across doses of 1.0 μM-7.5 μM of 3E10 (FIG. 1A).

In CD117+ cells isolated from bone marrow as well as from the spleens of treated mice, higher levels of gene editing were observed in animals treated with full length 3E10 plus PNA/DNA nanoparticles compared to animals treated with nanoparticles alone (FIGS. 1B and 1C).

### Example 3: 3E10 Enhances PNA/DNA Mediated Editing of the Beta Globin Gene in MEFs from a Mouse Model of Sickle Cell Disease

### Materials and Methods

### PNA and Donor DNA

The sequence of the PNA (designated tcPNA1A) was H-KKK-JJTJTTJ-OOO-CTTCTCCACAGGAGTCAG-KKK-NH₂ (SEQ ID NO:49) where the underlined nucleobases have a gamma mini-PEG side chain substitution, J-pseudoisocytosine, K=lysine, and O=flexible octanoic acid linker.

The single-stranded donor DNA oligomer was prepared by standard DNA synthesis and 5′ and 3′-end protected by inclusion of three phosphorothioate internucleoside linkages at each end. The sequence of the donor DNA was

(SEQ ID NO: 50)
5′TTGCCCCACAGGGCAGTAACGGCAGACTTCTCCTCAGGAGTCAG
GTGCACCATGGTGTCTGTTTG-3′.

### Mouse Model for Sickle Cells Disease

In sickle cell disease (SCD), the mutation (GAG->GTG) at codon 6 results in glutamic acid changed to valine. For correction (editing) of this SCD mutation site, studies were performed in the Townes mouse model.

The Townes mouse model was developed by Ryan T M, Ciavatta D J, Townes T M., “Knockout-transgenic mouse model of sickle cell disease.” Science. 1997 Oct. 31; 278 (5339): 873-6. PMID: 9346487.

Townes mice exclusively express human sickle hemoglobin (HbS). They were produced by generating transgenic mice expressing human α-, γ-, and β^{s}-globin that were then bred with knockout mice that had deletions of the murine α- and β-globin genes. Thus, the resulting progeny no longer express mouse α- and β-globin. Instead, they express exclusively human α- and β^{s}-globin. Hence, the mice express human sickle hemoglobin and possess many of the major hematologic and histopathologic features of individuals with SCD.

### Cell Culture and Treatment

Mouse embryonic fibroblasts (MEFs) were isolated from mouse embryos from a transgenic mouse model of sickle cell disease (Townes model, Jackson Laboratory). These MEFs were seeded in a 12-well plate at a seeding density of 200,000 cells per well. After 24 hours, cells were incubated with full length 3E10 (7.5 μM) for 5 minutes prior to the addition of 2 mg of nanoparticles per well. The nanoparticles contained either donor DNA alone or donor DNA plus tcPNA1A, which were designed to bind to and correct the beta globin gene at the site of the SCD mutation (A:T to T: A).

After 48 hours, the cells were washed 3 times prior to genomic DNA isolation (SV Wizard, Promega). Freshly isolated genomic DNA was analyzed by droplet digital PCR (ddPCR) to quantify gene editing frequencies.

### Results

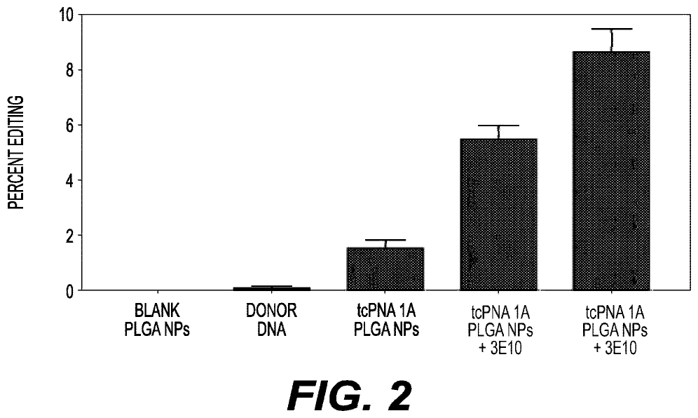

As shown in FIG. 2, untreated MEFs (blank controls) yielded no gene editing. Cells treated with PLGA NPs containing PNA/donor DNA achieved editing frequencies around 1% (FIG. 2). The addition of 3E10 prior to nanoparticle treatment substantially increased gene editing to 6%-8% (FIG. 2).

### Example 4: 3E10 Enhances PNA/DNA Mediated Editing of the Beta Globin Gene in BM Cells from a Mouse Model of Sickle Cell Disease

### Materials and Methods

### PNA and Donor DNA

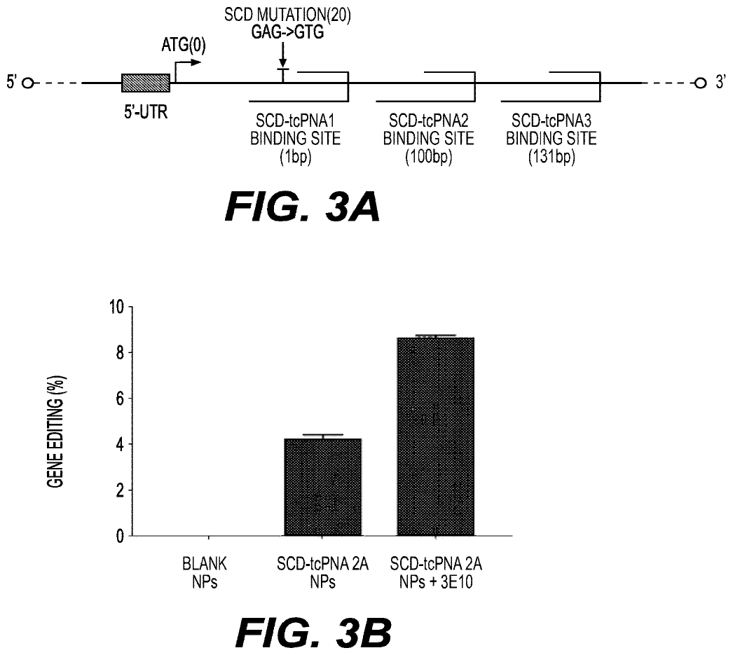

The sequence of the triplex forming PNA (designated tcPNA2A) was H-KKK-TTJJTJT-OOO-TCTCCTTAAACCTGTCTT-KKK-NH₂ (SEQ ID NO: 51) where the underlined nucleobases have a gamma mini-PEG side chain substitution, J=pseudoisocytosine, K=lysine, and O=flexible octanoic acid linker. The relative position of tcPNA2 in the beta globin locus is shown in FIG. 3A.

The single-stranded donor DNA oligomer was prepared by standard DNA synthesis and 5′ and 3′-end protected by inclusion of three phosphorothioate internucleoside linkages at each end. The sequence of the donor DNA was

(SEQ ID NO: 50)
5′TTGCCCCACAGGGCAGTAACGGCAGACTTCTCCTCAGGAGTCAG
GTGCACCATGGTGTCTGTTTG-3′.

### Cell Culture and Treatment

Bone marrow cells were isolated from the same transgenic mouse model of sickle cell disease described above in Example 3 (Townes model, Jackson Laboratory). Cells were treated with full length 3E10 plus 2 mg of nanoparticles per well. The nanoparticles contained the donor DNA plus tcPNA2A, designed to bind to and correct the beta globin gene at the site of the SCD mutation (A:T to T:A).

After 48 hours, the cells were washed prior to genomic DNA isolation (SV Wizard, Promega). Freshly isolated genomic DNA was analyzed by droplet digital PCR (ddPCR) to quantify gene editing frequencies.

### Results

To extend the findings observed in MEFs (described above in Example 3) to another cell type, the effect of 3E10 on gene editing in bone marrow cells was evaluated. As shown in FIG. 3B, untreated bone marrow cells (blank NPs) yielded no gene editing. Cells treated with PLGA NPs containing tcPNA2/donor DNA achieved editing frequencies around 4% (FIG. 3B). The addition of 3E10 prior to nanoparticle treatment substantially increased gene editing to more than 8% (FIG. 3B).

### Example 5: 3E10 Enhances PNA/DNA Mediated Editing In Vivo in the Townes Mouse Model

### Materials and Methods

To further validate whether 3E10 can boost gene editing in vivo, the Townes model (the same sickle cell transgenic mouse model used in Examples 3 and 4) was used. Mice were injected with a total of 4 doses of 2 mg of nanoparticles containing PNA/donor DNA over the course of 2 weeks, with the goal of correcting the codon 6 mutation in the beta globin gene. Three hours prior to each nanoparticle administration, mice were injected with 1 mg of 3E10 intraperitoneally (i.p). After two months, bone marrow cells were harvested and analyzed for editing via digital droplet PCR (ddPCR). Injections were performed every performed every 3 days over the course of 2 weeks as described above.

The sequence of the PNA used in these experiments, tcPNA1A, was H-KKK-JJTJTTJ-OOO-CTTCTCCACAGGAGTCAG-KKK-NH₂ (SEQ ID NO: 49) where the underlined nucleobases have a gamma mini-PEG side chain substitution, J-pseudoisocytosine, K=lysine, and O=flexible octanoic acid linker.

The sequence of the donor DNA was

(SEQ ID NO: 50)
5′TTGCCCCACAGGGCAGTAACGGCAGACTTCTCCTCAGGAGTCAG
GTGCACCATGGTGTCTGTTTG-3′.

### Results

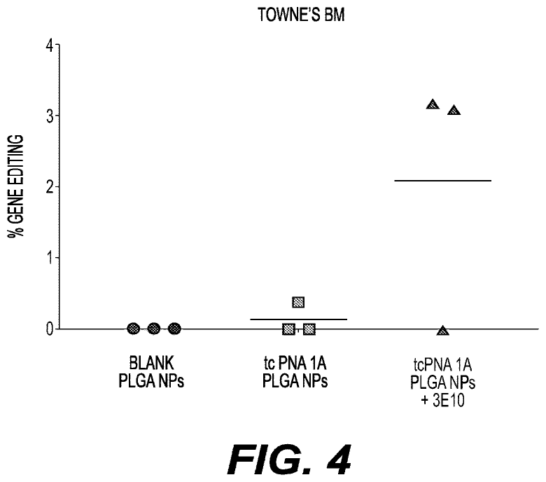

Compared to mice treated with nanoparticles alone, the addition of 3E10 substantially increased gene editing from an average editing frequency of 0.13% to 2.1% (FIG. 4).

### Example 6: 3E10 Enhances Beta Globin Editing in SC-1 Cells

### Materials and Methods

### PNA and Donor DNA

In the following experiments, NPs containing tcPNA2A was used. As previously described, the sequence of tcPNA2A is as follows: H-KKK-TTJJTJT-OOO-TCTCCTTAAACCTGTCTT-KKK-NH₂ (SEQ ID NO:51).

The sequence of the donor DNA was:

(SEQ ID NO: 50)
5′TTGCCCCACAGGGCAGTAACGGCAGACTTCTCCTCAGGAGTCAG
GTGCACCATGGTGTCTGTTTG-3′.

### Cell Culture and Treatment

SC-1 cells, a human lymphoblastoid cell line that carries the SCD mutation, were treated with 2 mg of nanoparticles per well with or without 3E10. After 48 hours, the cells were washed prior to genomic DNA isolation (SV Wizard, Promega). Freshly isolated genomic DNA was analyzed by droplet digital PCR (ddPCR) for editing frequencies.

### Results

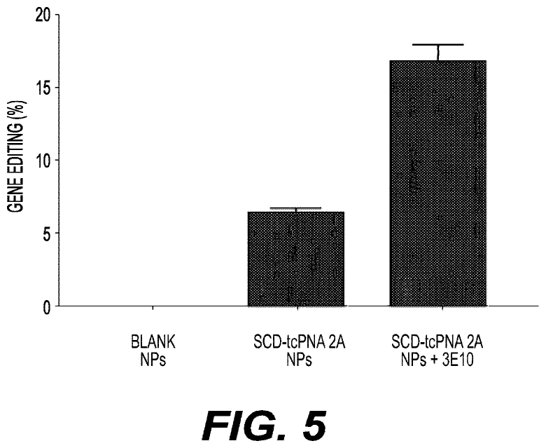

As shown in FIG. 5, blank controls yielded no gene editing. Cells treated with PLGA NPs containing tcPNA2A/Donor DNA achieved editing frequencies around 6%. The addition of 3E10 prior to nanoparticle treatment substantially increased gene editing to 17% (FIG. 5).

### Example 7: 3E10 Enhances Gene Editing by CRISPR/Cas9 Nickase Variant in K562 Cells

### Materials and Methods

K562 cells carrying a BFP/GFP reporter gene (Richardson, et al., Nat. Biotechnol., 34 (3): 339-44 (2016)) were transfected with CRISPR/Cas9 WT or CRISPR/Cas9 D10A nickase variant enzymes plus a guide RNA targeting the mutation site in GFP. Some samples were also treated with full-length 3E10, at a concentration of 1.5 mg/mL=10 μM.

Cas9 protein and guide RNAs were introduced by nucleofection as a ribonucleoprotein (RNP) complex. 45 pmol of Cas9 protein (D10A nickase variant or WT, both obtained from PNA Bio) with 45 pmol of sgRNA (synthesized with Invitrogen GeneArt kit) in Cas9 nuclease buffer (NEB), were pre-incubated for 5 minutes at room temperature.

Cells were nucleofected with the RNP complex and donor DNA having the sequence:

(SEQ ID NO: 52
GCCACCTACGGCAAGCTGACCCTGAAGTTCATCTGCACCACCGGC
AAGCTGCCGGTGCCCTGGCCCACCCTCGTGACCACCCTGACCTAC
GGCGTGCAGTGCTTCAGCCGCTACCCCGACCACATGA.

The sgRNA binding region was GCUGAAGCACUGCACGCCAU (SEQ ID NO:53).

The frequency of gene editing was measured two days later by flow cytometry for green fluorescence.

### Results

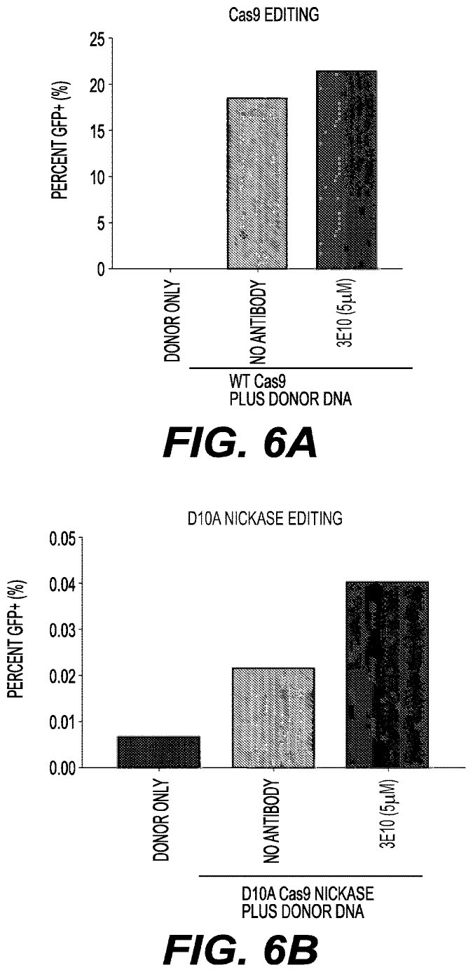

As shown in FIG. 6B, 3E10 treatment substantially boosted gene editing by the nickase Cas9 D10A.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A composition comprising
a gene editing technology selected from the group consisting of triplex-forming molecules, pseudocomplementary oligonucleotides, a CRISPR system, zinc finger nucleases (ZFN), transcription activator-like effector nucleases (TALEN), and intron encoded meganucleases, and
a potentiating agent that reduces one or more DNA repair pathways and increases genomic editing by the gene editing technology compared to the gene editing technology alone.

2. The composition of claim 1, wherein the potentiating agent is a cell-penetrating antibody, fragment or humanized variant thereof.

3. The composition of claim 2, wherein the cell-penetrating antibody is an anti-DNA antibody and inhibits RAD51.

4. The composition of claim 3, wherein the cell-penetrating antibody comprises a 3E10 monoclonal antibody or a cell-penetrating fragment thereof; a monovalent, divalent, or multivalent single chain variable fragment (scFv); or a diabody; or humanized form or variant thereof.

5. The composition of claim 4, comprising
(i) the CDRs of any one of SEQ ID NO:1-6, 12, or 13 in combination with the CDRs of any one of SEQ ID NO:7-11, or 15;
(ii) first, second, and third heavy chain CDRs selected from SEQ ID NO [S]: 15-23 in combination with first, second and third light chain CDRs selected from SEQ ID NO [S]: 24-30;
(iii) a humanized forms of (i) or (ii);
(iv) a heavy chain comprising an amino acid sequence comprising at least 85% sequence identity to any one of SEQ ID NO:1 or 2 in combination with a light chain comprising an amino acid sequence comprising at least 85% sequence identity to SEQ ID NO:7 or 8;
(v) a humanized form of (iv); or
(vi) a heavy chain comprising an amino acid sequence comprising at least 85% sequence identity to any one of SEQ ID NO:3-6 in combination with a light chain comprising an amino acid sequence comprising at least 85% sequence identity to SEQ ID NO:9-11.

6-9. (canceled)

10. The composition of claim 1, further comprising a donor oligonucleotide that induces a mutation(s) in the cell's genome by insertion or recombination induced or enhanced by the gene editing technology.

11-19. (canceled)

20. A pharmaceutical composition comprising the composition of claim 1 and a pharmaceutically acceptable excipient.

21. (canceled)

22. (canceled)

23. A method of modifying the genome of a cell comprising contacting the cell with a gene editing technology selected from the group consisting of triplex-forming molecules, pseudocomplementary oligonucleotides, a CRISPR system, zinc finger nucleases (ZFN), transcription activator-like effector nucleases (TALEN), and intron encoded meganucleases, and
a potentiating agent that reduces one or more DNA repair pathways and increases genomic editing by the gene editing technology compared to the gene editing technology alone.

24. The method of claim 23, wherein the gene editing technology and potentiating agent are part of different compositions.

25-34. (canceled)

35. The method of claim 23, wherein the cell's genome has a mutation underlying a disease or disorder selected from the group consisting of hemophilia, muscular dystrophy, globinopathies, cystic fibrosis, xeroderma pigmentosum, and lysosomal storage diseases, immune deficiency syndromes such as X-linked severe combined immunodeficiency and ADA deficiency, tyrosinemia, Fanconi anemia, the red cell disorder spherocytosis, alpha-1-anti-trypsin deficiency, Wilson's disease, Leber's hereditary optic neuropathy, and chronic granulomatous disorder.

36. The method of claim 35, wherein the mutation is in a gene encoding coagulation factor VIII, coagulation factor IX, dystrophin, beta-globin, CFTR, XPC, XPD, DNA polymerase eta, Fanconi anemia genes A through L, SPTA1 and other spectrin genes, ANK1 gene, SERPINA1 gene, ATP7B gene, interleukin 2 receptor gamma (IL2RG) gene, ADA gene, FAH gene, and genes linked to chronic granulomatous disease including the CYBA, CYBB, NCF1, NCF2, or NCF4 genes.

37. (canceled)

38. The method of claim 23, wherein the contacting occurs ex vivo.

39. The method of claim 38, wherein the cell is a hematopoietic stem cell.

40. The method of claim 23, further comprising administering a plurality of the cells to a subject in need thereof.

41. The method of claim 40, wherein the cells are administered to the subject in an effective amount to treat one or more symptoms of a disease or disorder.

42. The method of claim 23, wherein the contacting occurs in vivo following administration to a subject in need thereof.

43. The method of claim 42, wherein the subject has a disease or disorder selected from the group consisting of hemophilia, muscular dystrophy, globinopathies, cystic fibrosis, xeroderma pigmentosum, and lysosomal storage diseases, immune deficiency syndromes such as X-linked severe combined immunodeficiency and ADA deficiency, tyrosinemia, Fanconi anemia, the red cell disorder spherocytosis, alpha-1-anti-trypsin deficiency, Wilson's disease, Leber's hereditary optic neuropathy, and chronic granulomatous disorder.

44. The method of claim 43, wherein gene modification occurs in an effective amount to reduce one or more symptoms of the disease or disorder in the subject.

45. The method of claim 23, wherein the gene editing technology, potentiating agent, and optional donor oligonucleotide are encapsulated in nanoparticles together or separately.

46. (canceled)

47. (canceled)

48. The method of claim 45, wherein a targeting moiety, a cell penetrating peptide, or a combination thereof is associated with, linked, conjugated, or otherwise attached directly or indirectly to the nanoparticle.

49-54. (canceled)
